(19) United States
(12) Patent Application Publication
(10) Pub. No.: US 2024/0182633 A1
(43) Pub. Date: Jun. 6, 2024

(54) Incorporation of Carbon Dioxide Into Bioderived Polymer Scaffolds

(71) Applicant: Regents of the University of Minnesota, Minneapolis, MN (US)

(72) Inventors: Ian A. Tonks, Minneapolis, MN (US); Rachel Maria Rapagnani, Minneapolis, MN (US)

(21) Appl. No.: 18/548,777

(22) PCT Filed: Mar. 3, 2022
(86) PCT No.: PCT/US2022/018713

§ 371 Date: Sep. 1, 2023

Related U.S. Application Data
(60) Provisional application No. 63/156,135, filed on Mar. 3, 2021.

(51) Int. Cl. C08G 63/08; C07D 309/30; C08G 63/82; C08G 63/87; C08G 63/91

(52) U.S. Cl. CPC C08G 63/08; C07D 309/30; C08G 63/823; C08G 63/87; C08G 63/912

(57) ABSTRACT

Provided herein are methods of ring-opening polymerization of lactones to yield bioderived polyesters with varying backbone structures.

### CROSS-REFERENCE TO RELATED APPLICATIONS

This application claims the benefit of U.S. Application Ser. No. 63/156,135 entitled “INCORPORATION OF CARBON DIOXIDE INTO BIODERIVED POLYMER SCAFFOLDS” filed Mar. 3, 2021, which is incorporated by reference herein in its entirety.

### FEDERALLY SPONSORED RESEARCH OR DEVELOPMENT

This invention was made in part with United States Government support under contracts CHE-1901635 awarded by the Center for Sustainable Polymers, a part of the National Science Foundation. The U.S. Government has certain rights in the invention.

### TECHNICAL FIELD

This invention relates to ring-opening polymerization of lactones to yield bioderived polyesters with varying backbone structures.

### BACKGROUND

Carbon dioxide is inexpensive and abundant, and its prevalence as waste makes it attractive as a sustainable chemical feedstock. Although there are examples of copolymerizations of CO₂ with high-energy monomers, the direct copolymerization of CO₂ with olefins has not been reported. Given the challenges of direct conversion of CO₂ into polyesters, alternate strategies involving CO₂ conversion to inexpensive polymerizable intermediates are critically important. There is a need for a well-defined polyester derived solely from CO₂ and olefins, expanding access to new feedstocks.

### SUMMARY

This disclosure describes the use of renewable feedstocks (e.g., carbon dioxide from the atmosphere, butadiene from butanediol, and isoprene from rubber) to synthesize biodegradable polymers with hydrolytically cleavable ester linkages. The polymers can demonstrate amorphous/elastomeric behavior due at least in part to pendent alkyl groups. Reactions described herein help bypass barriers typically associated with the polymerization of carbon dioxide with olefins to make polyesters, one of the most widely used polymer classes. Mechanisms include ring-opening polymerization of intermediary lactone monomers synthesized from carbon dioxide and 1,3-dienes (e.g., one or both of butadiene and isoprene), which allows access to potentially biodegradable polyesters with bioderived backbones. Using renewable feedstocks to synthesize these materials can help mitigate plastic waste. The resulting polyesters can be used for a variety of applications, including drug delivery and as rubbery midblocks of sustainable thermoplastic elastomers.

Although the disclosed inventive concepts include those defined in the attached claims, it should be understood that the inventive concepts can also be defined in accordance with the following embodiments.

In addition to the embodiments of the attached claims and the embodiments described below, the following numbered embodiments are also innovative.

Embodiment 1 is a method of synthesizing a polyester, the method comprising: hydrogenating a lactone formed by telomerization of a first 1,3-diene, a second 1,3-diene, and carbon dioxide to yield a hydrogenated lactone; and polymerizing the hydrogenated lactone in the presence of a catalyst in a ring-opening process to yield a polyester. Embodiment 1 may provide one or more of the technical advantages as described herein.

Embodiment 2 is the method of embodiment 1, wherein the first 1,3-diene and the second 1,3-diene are the same.

Embodiment 3 is the method of embodiment 1 or 2, wherein the first 1,3-diene and the second 1,3-diene are independently butadiene, isoprene, or piperylene.

Embodiment 4 is the method of any one of embodiments 1 through 3, wherein the hydrogenated lactone is partially hydrogenated.

Embodiment 5 is the method of any one of embodiments 1 through 4, wherein the polyester comprises a pendent olefin.

Embodiment 6 is the method of any one of embodiments 1 through 5, further comprising crosslinking the polyester through the pendent olefin to yield a modified polyester.

Embodiment 7 is the method of embodiment 6, wherein crosslinking the polymer through the pendent olefin comprises reacting the pendent olefin with a multi-mercapto coupling agent.

Embodiment 8 is the method of embodiment 5, further comprising modifying the pendent olefin with a thiol-ene click reaction.

Embodiment 9 is the method of embodiment 8, wherein modifying the pendent olefin with the thiol-ene click reaction comprises functionalizing the polyester with a carboxylic acid.

Embodiment 10 is the method of embodiment 8, wherein modifying the pendent olefin with the thiol-ene click reaction comprises functionalizing the polyester with a tertiary amine.

Embodiment 11 is the method of embodiment 10, further comprising quaternizing the amine to yield a modified polyester with antibacterial properties.

Embodiment 12 is the method of any one of embodiments 1 through 11, wherein the catalyst is an acid catalyst or an organocatalyst.

Embodiment 13 is the method of any one of embodiments 1 through 12, wherein the catalyst comprises one or more of diphenyl phosphate, sodium methoxide, triazabicyclodecene, bistriflimide, methane-sulfonic acid, and diaza-bicycloundecene.

Embodiment 14 is the method of any one of embodiments 1 through 13, wherein the polymerizing occurs at a temperature between about 0° C. and about 166° C.

Embodiment 15 is the method of any one of embodiments 1 through 14, further comprising synthesizing the lactone.

Embodiment 16 is the method of embodiment 15, wherein synthesizing the lactone comprises capturing carbon dioxide from the atmosphere.

Embodiment 17 is the polyester of any one of embodiments 1 through 16.

Embodiment 18 is the polyester of embodiment 17, wherein the polyester is represented by one of the following structures:

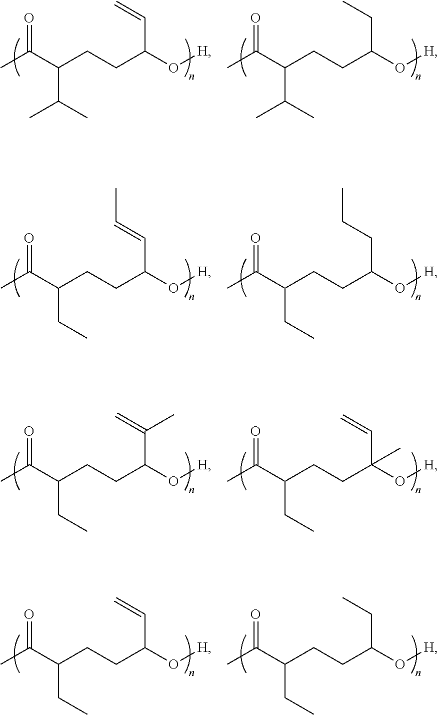

wherein n is an integer.

Embodiment 19 is the polyester of embodiment 18, wherein n is in a range between about 2 and about 200.

Embodiment 20 is the modified polyester of any one of embodiments 6 through 16.

Embodiment 21 is a method of synthesizing a lactone, the method comprising: telomerizing piperylene with carbon dioxide to yield the lactone.

Embodiment 22 is the method of embodiment 21, wherein the lactone is represented by one of the following structures:

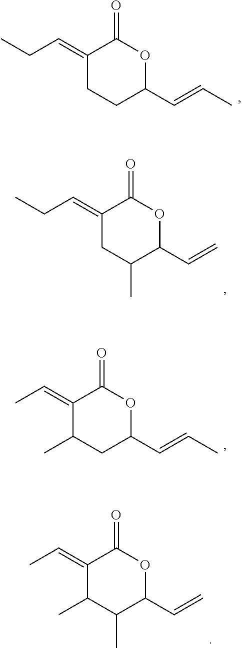

Embodiment 23 is the lactone of embodiment 21 or 22.

Embodiment 24 is a method of synthesizing a polyester, the method comprising: hydrogenating a lactone formed by the method of embodiment 21 to yield a hydrogenated lactone; and polymerizing the hydrogenated lactone in the presence of a catalyst in a ring-opening process to yield a polyester.

The details of one or more embodiments of the subject matter of this disclosure are set forth in the accompanying drawings and the description. Other features, aspects, and advantages of the subject matter will become apparent from the description, the drawings, and the claims.

### BRIEF DESCRIPTION OF DRAWINGS

FIG. 1 depicts reaction pathways for palladium-catalyzed telomerization of carbon dioxide and 1,3-dienes (e.g., butadiene and isoprene) to yield lactone monomers, and the subsequent organo- or acid-catalyzed ring-opening polymerization of the lactone monomers to yield bioderived, biodegradable polyesters with varying backbone structures, including those with pendent olefins which can be further functionalized.

FIG. 2 depicts reaction pathways to polymer involving butadiene/piperylene/CO₂ co-telomerization.

FIG. 3 depicts telomerization of piperylene and CO₂.

FIG. 4 shows a ¹H NMR spectrum in CDCl₃ for the polymerization depicted in FIG. 1 catalyzed by diphenyl phosphate.

FIG. 5 depicts a reaction pathway for the addition of a carboxylic acid group to polymer P-I of FIG. 1.

FIG. 6 depicts addition of a quaternary amine to polymer P-1 of FIG. 1.

FIG. 7A depicts photoinitiated crosslinking of polymer P-1 of FIG. 1.

FIG. 7B depicts crosslinking of polymer P-1 of FIG. 1 using multi-mercapto coupling agents.

FIG. 8 depicts polymerization results of a reaction including 3-ethyl-6-vinyltetrahydro-2H-pyran-2-one (EtVP), 1 mol % phenylpropanol (PPA) initiator, and 5 mol % catalyst over 72 h using various catalysts.

FIG. 9A is a graph of the size-exclusion chromatography differential refractive index of poly(3-ethyl-6-vinyltetrahydro-2H-pyran-2-one) (poly(EtVP)).

FIG. 9B is a graph of the thermogravimetric analysis of poly(EtVP).

FIG. 9C is a graph of the differential scanning calorimetry of poly(EtVP).

FIG. 10A is a graph of the size-exclusion chromatography differential refractive index of poly(3,6-diethyl-tetrahydro-2H-pyran-2-one) (poly(DEP)).

FIG. 10B is a graph of the thermogravimetric analysis of poly(DEP).

FIG. 10C is a graph of the differential scanning calorimetry of poly(DEP).

FIG. 11 is a graph of R*ln(nm/no) vs. 1000/T for EtVP and DEP (van′t Hoff analysis), where nm is the moles of monomer at equilibrium and no is the initial moles of monomer.

FIG. 12A is a kinetic plot of the EtVP polymerization with 5 mol % 1,5,7-triazabicyclo[4.4.0]dec-5-ene (TBD) catalyst.

FIG. 12B is a kinetic plot of the EtVP polymerization with 5 mol % diketopyrrolopyrrole (DPP) catalyst.

FIG. 12C is a kinetic plot of the EtVP polymerization with 10 mol % NaOMe catalyst.

FIG. 12D is all three kinetic plots of the EtVP polymerization overlaid.

FIG. 13A is a graph of the size-exclusion chromatography differential refractive index of poly(EtVP-b-PLA), wherein PLA is polylactide.

FIG. 13B is a graph of the thermogravimetric analysis of poly(EtVP-b-PLA).

FIG. 13C is a graph of the differential scanning calorimetry of poly(EtVP-b-PLA).

FIG. 14A is a graph of the thermogravimetric analysis of the quarternization of poly(EtVP-DAT) with benzyl bromide, wherein DAT is 2-diethylaminoethanethiol.

FIG. 14B is a graph of the differential scanning calorimetry of the quarternization of poly(EtVP-DAT) with benzyl bromide.

FIG. 15A is a graph of the size-exclusion chromatography differential refractive index of poly(EtVP-BMP), wherein BMP is butyl-3-mercaptopropionate.

FIG. 15B is a graph of the thermogravimetric analysis of poly(EtVP-BMP).

FIG. 15C is graph of the differential scanning calorimetry of poly(EtVP-BMP).

FIG. 16A is a graph of the degradation of poly(EtVP) in 0.1 M NaOH.

FIG. 16B is a graph of the degradation of poly(EtVP) in 0.1 M HCl.

FIG. 16C is a graph of the degradation of poly(EtVP in 0.01 M phosphate-buffered saline.

FIG. 17A is a graph of the hydrolytic degradation of poly(EtVP).

FIG. 17B is a graph of the CO₂ respirometry data showing biodegradation of poly(EtVP) under aqueous aerobic conditions.

FIG. 18 depicts various post-polymerization modifications of poly(EtVP).

### DETAILED DESCRIPTION

Provided herein are methods of synthesizing a polyester. In some embodiments, the method includes hydrogenating a lactone formed by telomerization of a first 1,3-diene, a second 1,3-diene, and carbon dioxide to yield a hydrogenated lactone, and polymerizing the hydrogenated lactone in the presence of a catalyst in a ring-opening process to yield a polyester.

In some embodiments, the first 1,3-diene and the second 1,3-diene are the same. In some embodiments, the first 1,3-diene and the second 1,3-diene are different. In some embodiments, the first 1,3-diene and the second 1,3-diene are independently butadiene, isoprene, or piperylene.

In some embodiments, the hydrogenated lactone is partially hydrogenated. For example, the partially hydrogenated lactone is 3-ethyl-6-vinyltetrahydro-2H-pyran-2-one (EtVP). In some embodiments, the hydrogenated lactone is fully hydrogenated. For example, the fully hydrogenated lactone is 3,6-diethyltetrahydro-2H-pyran-2-one (DEP).

In some embodiments, the polyester comprises a pendent olefin. For example, the polyester is poly(EtVP). In some embodiments, the pendent olefin is used for crosslinking the polyester. In some embodiments, the pendent olefin is used for modifying the polyester.

In some embodiments, the method further includes crosslinking the polyester through the pendent olefin to yield a modified polyester. In some embodiments, crosslinking the polymer through the pendent olefin comprises reacting the pendent olefin with a multi-mercapto coupling agent.

In some embodiments, the method further includes modifying the pendent olefin with a thiol-ene click reaction. In some embodiments, modifying the pendent olefin with the thiol-ene click reaction includes functionalizing the polyester with a carboxylic acid. In some embodiments, modifying the pendent olefin with the thiol-ene click reaction comprises functionalizing the polyester with a tertiary amine.

In some embodiments, the method further includes quaternizing the amine to yield a modified polyester with antibacterial properties.

In some embodiments, the catalyst is an acid catalyst or an organocatalyst. In some embodiments, the catalyst is an acid catalyst. In some embodiments, the catalyst is an organocatalyst. In some embodiments, the catalyst includes one or more of diphenyl phosphate, sodium methoxide, triazabicyclodecene, bistriflimide, methane-sulfonic acid, and diaza-bicycloundecene. In some embodiments, the catalyst includes triazabicyclodecene. In some embodiments, the catalyst includes diaza-bicycloundecene.

In some embodiments, the polymerizing occurs at a temperature between about 0° C. and about 166° C., or about 0° C. to about 100° C., or about 0° C. and about 80° C., or about 10° C. and about 50° C., or about room temperature. In some embodiments, the polymerizing occurs at room temperature.

In some embodiments, the method further comprises synthesizing the lactone. In some embodiments, synthesizing the lactone comprises capturing carbon dioxide from the atmosphere.

In some embodiments, the polyester is represented by one of the following structures:

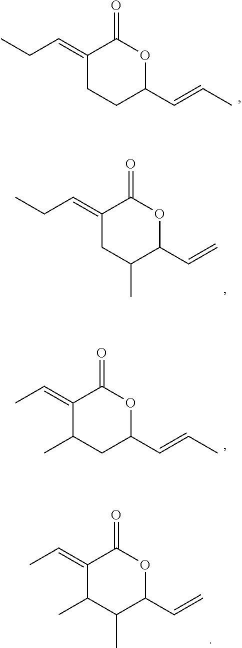

wherein n is an integer. In some embodiments, n is an integer in a range between about 2 and about 200, or about 10 to about 200, or about 50 to 200, or about 100 to about 200.

Also provided herein is a method of synthesizing a lactone. The method includes telomerizing piperylene with carbon dioxide to yield the lactone. In some embodiments, the lactone is represented by one of the following structures:

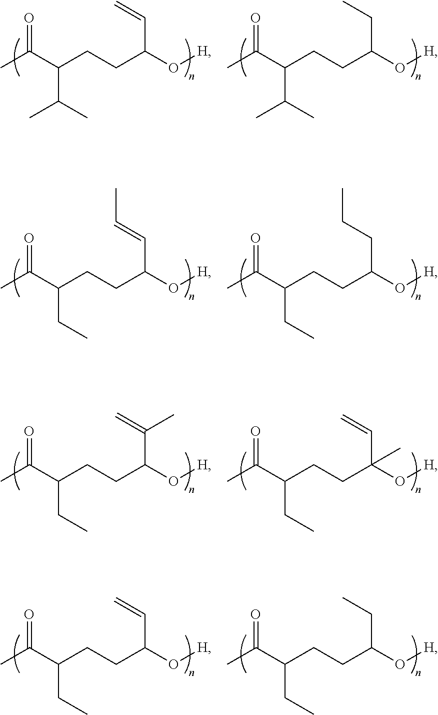

Also provided herein are methods of synthesizing a polyester, wherein the method includes hydrogenating a lactone formed by the method of synthesizing a lactone disclosed above to yield a hydrogenated lactone, and polymerizing the hydrogenated lactone in the presence of a catalyst in a ring-opening process to yield a polyester.

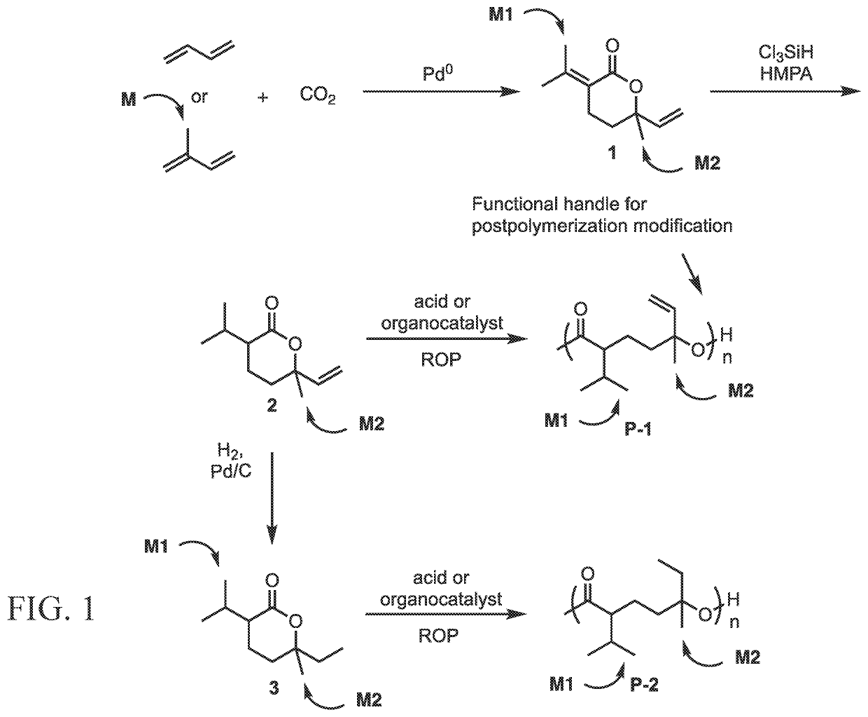

FIG. 1 depicts reaction pathways for palladium-catalyzed telomerization of carbon dioxide and 1,3-dienes (e.g., butadiene and isoprene) to synthesize lactone monomers. When two isoprene molecules with methyl group M react with carbon dioxide to yield 1, the resulting lactone includes methyl groups M1 and M2. When an isoprene molecule and a butadiene molecule react with carbon dioxide to yield 1, the resulting lactone includes one of methyl groups M1 and M2. When two butadiene molecules react with carbon dioxide to yield 1, the resulting lactone does not include methyl groups M1 and M2.

In one example, the telomerization of butadiene and CO₂ was carried out in a 300 mL Parr reactor. The reactor was charged with a solution of Pd(dba)₂ and P(o-OMePh)₃ in acetonitrile, which was followed by the addition of freshly condensed butadiene. The reactor was then sealed, purged with nitrogen 3 times, and charged with 450 psi CO₂. This was allowed to stir at 80 C for ˜22 hours, then the vessel was cooled to RT and vented. The resulting yellow liquid was filtered through silica then concentrated under reduced pressure. Column chromatography and distillation were carried out to purify the lactone.

Lactone 1 is hydrogenated (e.g., in trichlorosilane (Cl₃SiH) and hexamethylphosphoramide (HMPA)) to yield selectively hydrogenated monomer 2.

In one example, the hydrogenation of lactone 1 was performed on a Schlenk line. First, lactone 1 and HMPA were dissolved in dry DCM. The reaction mixture was then cooled to 0° C. and Cl₃SiH was slowly added. This was allowed to stir overnight, then the reaction was quenched with saturated sodium bicarbonate and diluted with ethyl acetate. This was filtered through Celite, then was transferred to a separatory funnel, washed with water 3× and brine 1×, then the organic phase was concentrated under reduced pressure.

The lactone monomers then undergo subsequent organo- or acid-catalyzed ring-opening polymerization to yield bioderived, biodegradable polyesters. In some implementations, selectively hydrogenated monomer 2 undergoes ring-opening polymerization in the presence of an acid catalyst or organocatalyst to yield a polyester P-1. The polyester P-1 includes a pendent olefin that can be for further reactions to yield a modified polyester, such as functionalization (e.g., with a thiol-ene click reaction) and crosslinking (e.g., with multi-mercapto coupling agents). Functionalization and crosslinking can be selected to yield modified polyesters with specific properties. In some implementations, lactone 1 undergoes further hydrogenation (e.g., with hydrogen gas in the presence of palladium on carbon) to yield fully hydrogenated monomer 3. Monomer 3 can undergo a ring-opening polymerization in the presence of an acid catalyst or organocatalyst to yield a polyester P-2.

Suitable catalysts for ring-opening polymerizations include diphenyl phosphate, sodium methoxide, triazabicyclodecene, bistriflimide, methane-sulfonic acid, and diaza-bicycloundecene.

Incorporation of Piperylene into Lactones

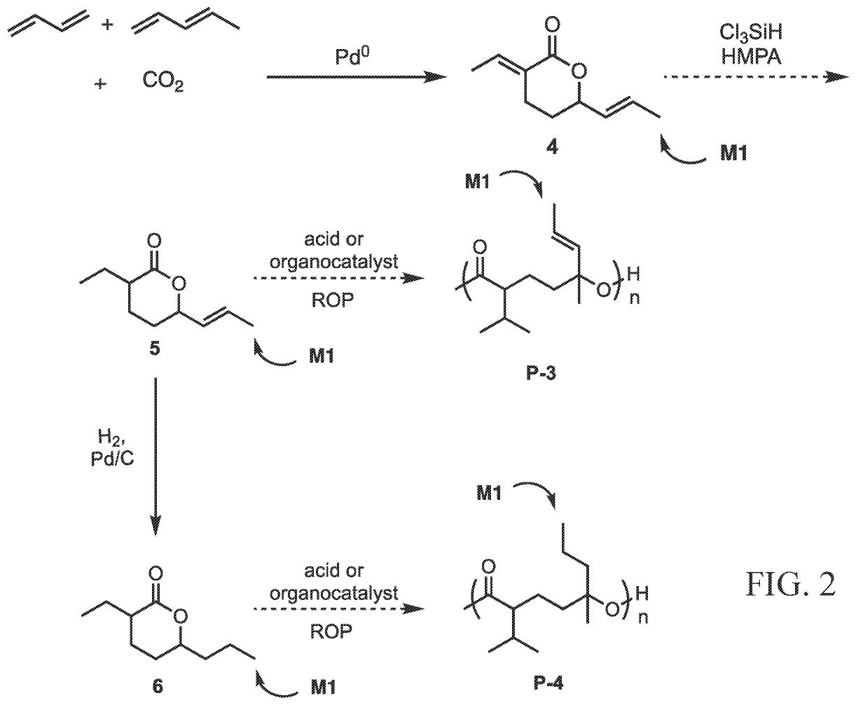

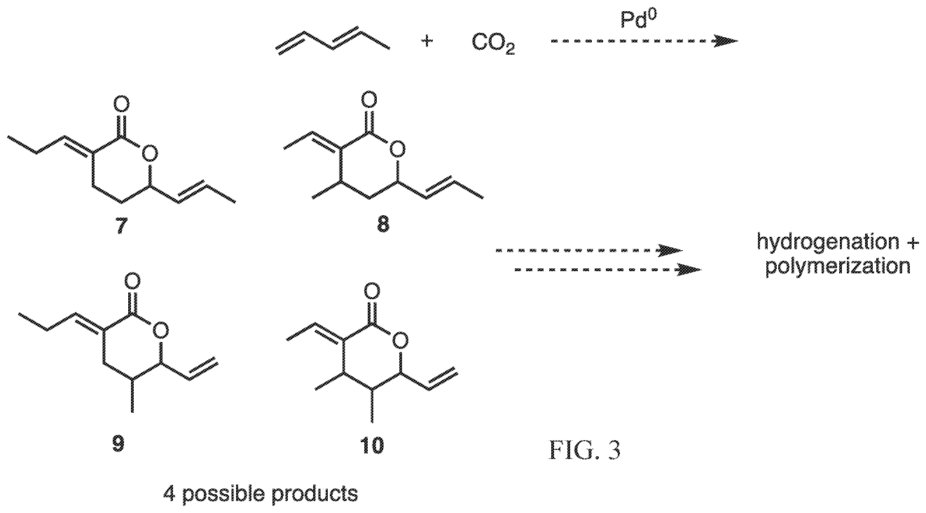

Other 1,3-dienes can be used to synthesize lactone monomers in reactions similar to those described with respect to FIG. 1. Examples of suitable dienes include piperylene, 1,3-hexadiene, and myrcene. As depicted in FIG. 2, piperylene can be incorporated into lactone monomers, and thus the resulting polyesters. The piperylene methyl group is identified as M1. The selectively and fully hydrogenated lactone monomers 5 and 6, respectively, are formed in reactions similar to those described with respect to selectively and fully hydrogenated lactone monomers 2 and 3 in FIG. 1. FIG. 3 depicts telomerization of piperylene and CO₂ to yield lactone monomers 7-10, which can undergo hydrogenation and polymerization in reactions similar to those described with respect to FIG. 1.

### EXAMPLES

Materials and Methods

Solvents and reagents were purchased from Sigma-Aldrich, STREM, Oakwood Chemicals, Matheson, and Airgas and were used without further purification unless otherwise noted. Deuterated chloroform (CDCl₃) was purchased from Cambridge Isotope Laboratories and used without further purification. All polymerizations were carried out in a nitrogen-filled glovebox (VAC) unless otherwise specified. Flash column chromatography was performed on a Teledyne ISCO Combiflash NextGen 300+@ with 40 g of silica RediSep® normal-phase silica flash columns. High-resolution electrospray mass spectrometry (ESI-MS) was performed on all isolated samples using a Bruker BioTOF II ESI/TOF-MS with PEG 200 as an internal mass standard. Size exclusion chromatography (SEC) was performed in tetrahydrofuran (THF) using an Agilent 1260 Infinity LC system equipped with three Waters Styragel columns (HR6, HR4, HR1, 5 μm particles of poly(styrene-divinylbenzene)) connected in series and fitted with a Wyatt OPTILAB T-rEX refractive index detector at 25° C. and a flow rate of 1 mL/min. Determination of molar masses and dispersities was made by calibration against polystyrene standards. Thermogravimetric analyses (TGA) were performed on a TA Instruments Q500 under a nitrogen atmosphere at a heating rate of 10° C./min). Differential scanning calorimetry (DSC) analysis was performed on a TA Instruments Discovery DSC using hermetically sealed aluminum T-zero pans. Scans were conducted under a nitrogen atmosphere at a heating/cooling rate of 5° C./min unless otherwise noted. ¹H NMR and ¹³C NMR spectra were recorded on Bruker Avance III HD 400 MHz and Bruker Avance III 500 MHz spectrometers. ¹H NMR spectra of all polymers were run with a relaxation delay of 10 seconds unless otherwise noted. Chemical shifts are reported with respect to tetramethylsilane (TMS). OECD-301B biodegradation studies were conducted by Situ Biosciences.

### Example 1: Synthesis of 3-ethylidene-6-vinyltetrahydro-2H-pyran-2-one (EVP)

Procedure adapted from Sharif et. al. ChemCatChem 2017, 9, 542-546. The telomerization of butadiene and CO₂ was carried out in a 300 mL bomb reactor. The reactor was charged with a solution of Pd(dba)₂ (163.3 mg, 0.284 mmol, 0.0005 eq.) and P(o-OMePh)₃ (300.57 mg, 0.853 mmol, 0.0015 eq.) in acetonitrile (130 mL) and a stir bar, which was followed by the addition of freshly condensed butadiene (50 mL, 568.5 mmol, 1 eq.). The reactor was then sealed, cooled to approximately −10 or −20° C., purged with nitrogen 2 times, warmed to room temperature, and charged with 450 psi CO₂. The reactor was then heated to 80° C. and was allowed to stir for 22 hours. The reactor was cooled in an ice bath and vented. The resulting liquid was filtered through silica then concentrated under reduced pressure. Column chromatography (5:1 hexanes: ethyl acetate) and distillation were carried out to obtain 11.05 g clear liquid (25.5% yield). ¹H NMR (400 MHz, CDCl₃) δ 7.18-7.11 (m, 1H), 5.89 (m, 1H), 5.23-5.38 (dd, 2H), 4.78 (m, 1H), 2.64-2.39 (m, 2H), 2.10-2.03 (m, 1H), 1.82-1.75 (m, 4H); ¹³C NMR (101 MHz, CDCl₃) δ 166.26, 141.16, 135.87, 125.98, 116.89, 78.94, 27.64, 21.99, 14.12.

### Example 2: Synthesis of 3-ethyl-6-vinyltetrahydro-2H-pyran-2-one (EtVP)

Procedure adapted from Sugiura et. al., Chem. Commun. 2008, 2, 4309-4311. EVP (4.52 g, 27.2 mmol, 1 eq.) and hexamethylphosphoramide (HMPA) (1.05 mL, 5.94 mmol, 0.2 eq.) were added to a flame-dried 500 mL Schlenk flask and were dissolved in anhydrous dichloromethane (DCM) (dried by passing through activated alumina columns of a Pure Process Technology solvent purification system) (60 mL, 0.5 M). The reaction mixture was then cooled to 0° C. and Cl₃SiH (6 mL, 59.4 mmol, 2 eq.) was added dropwise. The reaction mixture was allowed to stir between 4 and 16 hours, then the reaction was quenched slowly with saturated sodium bicarbonate (100 mL) and diluted with ethyl acetate (100 mL). This was filtered through Celite then was transferred to a separatory funnel. The organic phase was washed 3 times with H₂O and 1 time with brine, dried with Na₂SO₄, then concentrated under reduced pressure to give 3.93 g liquid (86% yield). Vacuum distillation was performed to remove residual butadiene dimer (from EVP synthesis) if necessary. ¹H NMR (400 MHz, CDCl₃) δ 5.91-5.81 (m, 1H), 5.33 (d, 1H), 5.22 (t, 1H), 4.84-4.73 (m, 1H), 2.44-2.33 (m, 1H), 2.10-1.46 (m, 6H), 1.00-0.95 (m, 3H); ¹³C NMR (101 MHz, CDCl₃) δ 174.69, 173.36, 136.62, 136.09, 117.01, 116.70, 81.10, 78.29, 42.02, 40.37, 28.91, 27.13, 24.94, 24.61, 24.13, 22.51, 11.53, 11.19. ESI-HRMS (m/z): calcd. for C₉H₁₄O₂Na⁺, 177.0886; found, 177.0885 (diff. 0.0001).

### Example 3: Synthesis of 3,6-diethyltetrahydro-2H-pyran-2-one (DEP)

Procedure adapted from Mango and Lenz, Die Makromol. Chemie 1973, 163, 13-36. EtVP (4.0 g, 25.6 mmol, 1 eq.) was added to a 250 mL 3-neck flask equipped with a stir bar and a reflux condenser, followed by o-xylene (75 mL, 0.34 M). This was heated to 150° C., and at the onset of reflux, p-toluenesulfonyl hydrazide (9.7 g, 52 mmol, 2 eq.) was added. After 4 hours, the solution turned an orange color. The solution was then cooled down and vacuum filtered through a fine frit packed with Celite. The filtrate was concentrated under reduced pressure then further purified by vacuum distillation to obtain 3.12 g liquid (77% yield). ¹H NMR (400 MHz, CDCl₃) δ 4.20-4.13 (m, 1H), 2.39-2.27 (m, 1H), 2.08-1.37 (m, 8H), 0.98-0.91 (m, 6H); ¹³C NMR (101 MHz, CDCl₃) δ 175.79, 173.93, 82.60, 79.34, 42.14, 39.73, 29.18, 28.32, 26.28, 24.90, 23.89, 22.92, 11.63, 11.09, 9.61, 9.27.

### Example 4: Polyester Synthesis from 3-ethyl-6-vinyltetrahydro-2H-pyran-2-one (EtVP)

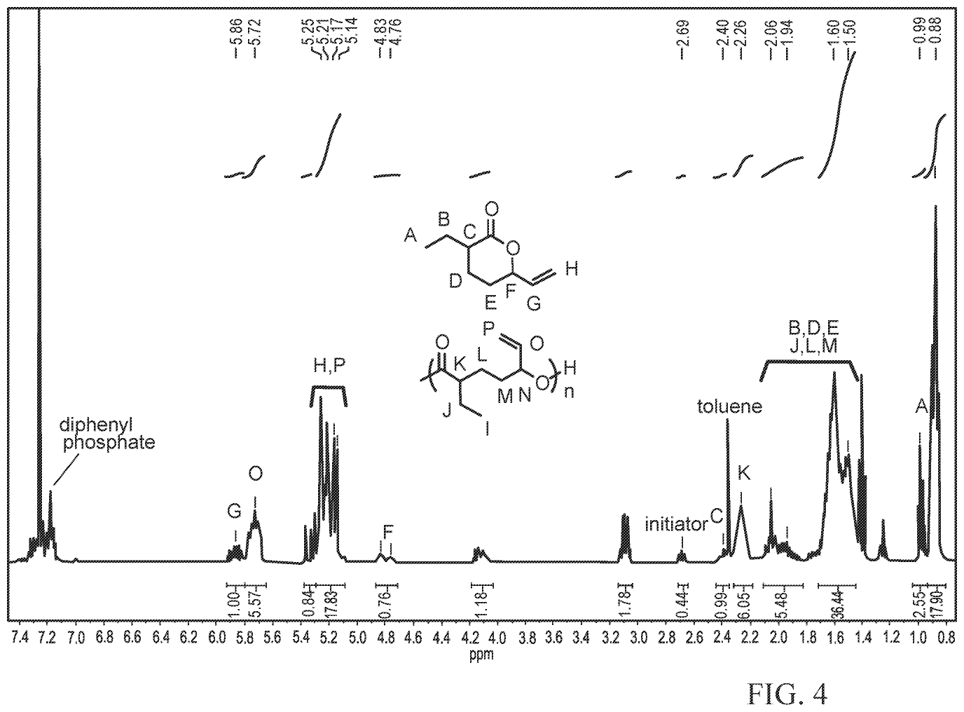

In an N₂ glovebox, lactone 2 (203.6 mg, 1.32 mmol) was added to a one-dram vial equipped with a stir bar. This was followed by the addition of diphenyl phosphate (12.3 mg, 0.05 mmol) and 3-phenyl-1-propanol (5 μL, 0.037 mmol). The sealed vial was removed from the glovebox and allowed to stir at room temperature for 55 days, with NMR aliquots being removed on day 4, 11, 23, and 37. On day 55, the polymerization was quenched with a few drops of triethylamine, then using toluene the material was transferred to a new vial. After concentrating under reduced pressure, 170 mg viscous, colorless oil was obtained. By ¹H NMR the conversion was determined to be 84.7%, and by end group analysis Mₙ was calculated to be approximately 4,000 g/mol. ¹H NMR (CDCl₃, 400 MHz): ¹H NMR (CDCl₃, 400 MHz): 5.92-5.83 (m, 1H, monomer), 5.77-5.67 (m, 1H, polymer), 5.34 (dt, 1H, monomer), 5.26-5.14 (m, 1H, monomer & polymer), 4.86-4.73 (m, 1H, monomer), 2.69 (t, 2H, initiator), 2.44-2.34 (m, 1H, monomer), 2.27 (broad m, 1H, polymer), 2.05-1.41 (m, 6H, monomer & polymer), 1.01-0.96 (m, 3H, monomer), 0.88 (t, 3H, polymer). FIG. 4 shows a ¹H NMR spectrum in CDCl₃ for DPP-catalyzed reaction 1.

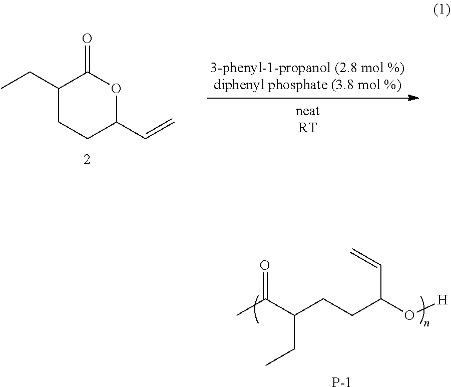

In air, lactone 2 (206.8 mg, 1.341 mmol) was added to a one-dram vial equipped with a stir bar. This was followed by methanesulfonic acid (2 μL, 0.031 mmol) and 3-phenyl-1-propanol (4 μL, 0.029 mmol). The vial was allowed to stir at room temperature for 36 days, with NMR aliquots being removed on day 1, 5, 9, and 20. On day 36, the polymerization was quenched with a few drops of triethylamine, then using excess toluene the material was transferred to a new vial. After concentrating under reduced pressure, 160 mg viscous, slightly yellow oil was obtained. By ¹H NMR the conversion was determined to be 82%, and by end group analysis Mₙ was calculated to be approximately 2,600 g/mol. ¹H NMR (CDCl₃, 400 MHz): 5.92-5.82 (m, 1H, monomer), 5.77-5.69 (m, 1H, polymer), 5.34 (dt, 1H, monomer), 5.25-5.14 (m, 1H, monomer & polymer), 4.86-4.73 (m, 1H, monomer), 2.69 (t, 2H, initiator), 2.44-2.34 (m, 1H, monomer), 2.27 (broad m, 1H, polymer), 2.09-1.41 (m, 6H, monomer & polymer), 1.01-0.96 (m, 3H, monomer), 0.89 (t, 3H, polymer)

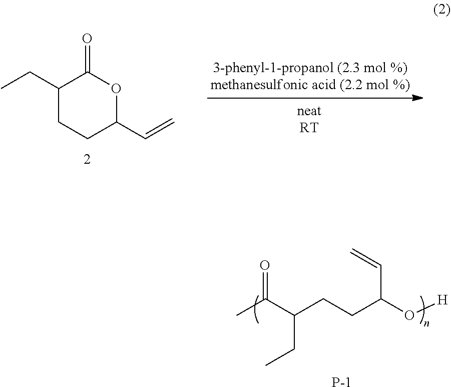

In an N₂ glovebox, lactone 2 (204.7 mg, 1.327 mmol) was added to a one-dram vial equipped with a stir bar. This was followed by 1,5,7-triazabicyclo[4.4.0]dec-5-ene (6 mg, 0.043 mmol) and 3-phenyl-1-propanol (5 μL, 0.037 mmol). The sealed vial was removed from the glovebox and allowed to stir at room temperature for 51 days, with NMR aliquots being removed on day 3, 6, 12, 20, and 34. On day 51, the polymerization was quenched with a few drops of 6 M acetic acid in toluene, then using excess toluene the material was transferred to a new vial. After concentrating under reduced pressure, 190 mg viscous, colorless oil was obtained. By ¹H NMR the conversion was determined to be 85%, and by end group analysis Mₙ was calculated to be approximately 3,500 g/mol. ¹H NMR (CDCl₃, 400 MHz): 5.92-5.83 (m, 1H, monomer), 5.77-5.67 (m, 1H, polymer), 5.34 (dt, 1H, monomer), 5.26-5.14 (m, 1H, monomer & polymer), 4.86-4.73 (m, 1H, monomer), 2.69 (t, 2H, initiator), 2.44-2.34 (m, 1H, monomer), 2.27 (broad m, 1H, polymer), 2.06-1.41 (m, 6H, monomer & polymer), 1.01-0.96 (m, 3H, monomer), 0.89 (t, 3H, polymer).

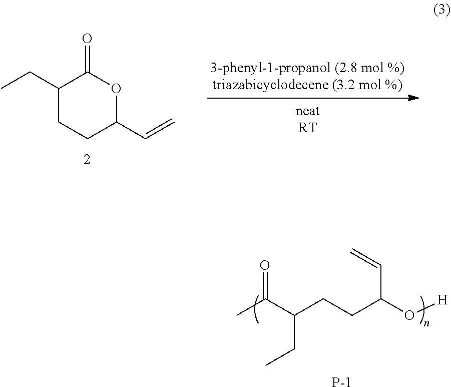

Temperature Range for Catalysis: Performing van′t Hoff analysis on the polymerization of the selectively hydrogenated butadiene/CO₂-derived lactone provided information about the thermodynamic parameters. From this analysis, the ΔHₚ was determined to be −3.07 kcal/mol and the ΔSₚ was determined to be −7 cal/K·mol. With these values, the ceiling temperature—the temperature at which no polymerization occurs at equilibrium—was calculated by dividing ΔHₚ by ΔSₚ. For this polymerization the ceiling temperature is 166° C., which falls between the ceiling temperatures of poly(∂-decalactone) and poly(∂-hexalactone). The equilibrium temperature—the temperature at which the monomer conversion reaches 50%—was also calculated, which is 94° C. for this polymerization. A polymerization can be run well below its ceiling temperature/equilibrium temperature (e.g., as low as 5° C.) in order to reach good (>80-90%) conversions.

The lowest temperature at which this polymerization was performed is 5° C. with sodium methoxide as the initiator/catalyst. The polymerization was quenched after 50 days and the resulting material was shown by ¹H NMR to have reached 84.6% conversion and an Mₙ of approximately 3,750 g/mol.

### Example 5: Post Polymerization Modification

Thiol-ene click chemistry can be used for post-polymerization modification. Polymers can be imparted with different properties and applications depending on the thiol used. This kind of versatility is an attractive feature, especially from a bioderived monomer. The following examples are two of many pendent groups that can be used to control the properties and thus applications of polymer P-1.

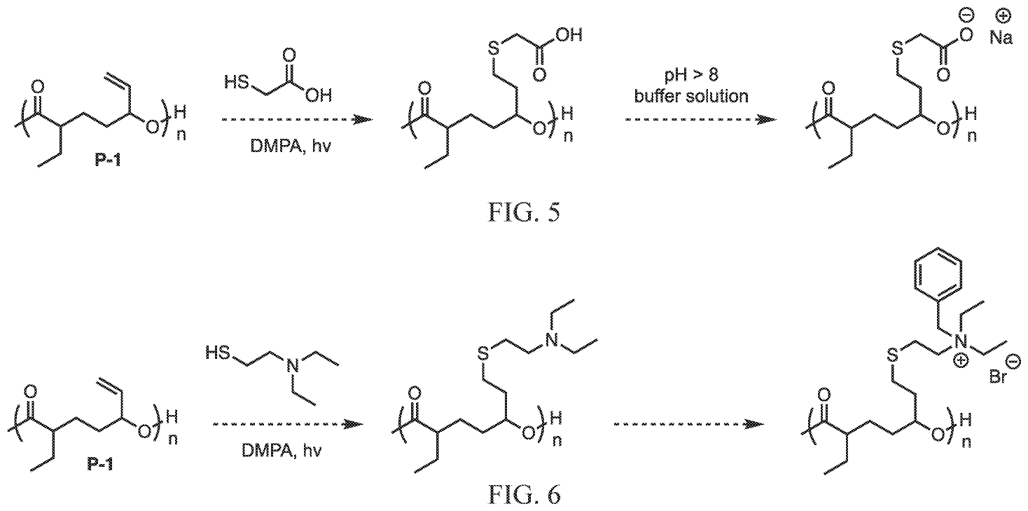

As depicted in FIG. 5, a carboxylic acid can react with polymer P-1 to increase water solubility of the resulting polymer, based at least in part on the pH of solution. This reaction can be used advantageously to yield polyesters for biomedical applications or for degradation in aquatic environments.

Another modification of polymer P-1, as depicted in FIG. 6, is the addition of a thiol with a tertiary amine moiety followed by quaternization of the amine, with the resulting quaternary amine imparting antimicrobial properties to the resulting polyester. The percentage of amine functionality can be modulated to yield a water-insoluble polymer capable of interacting with the cell membrane of the bacteria.

In pursuing this type of post-polymerization modification, a test reaction was performed on lactone 2 using 1-butanethiol, DMPA, and UV light. After 30 minutes, the pendent alkene peaks had disappeared in the ¹H NMR, showing that this reaction has the potential to be translated to the modification of this polymer.

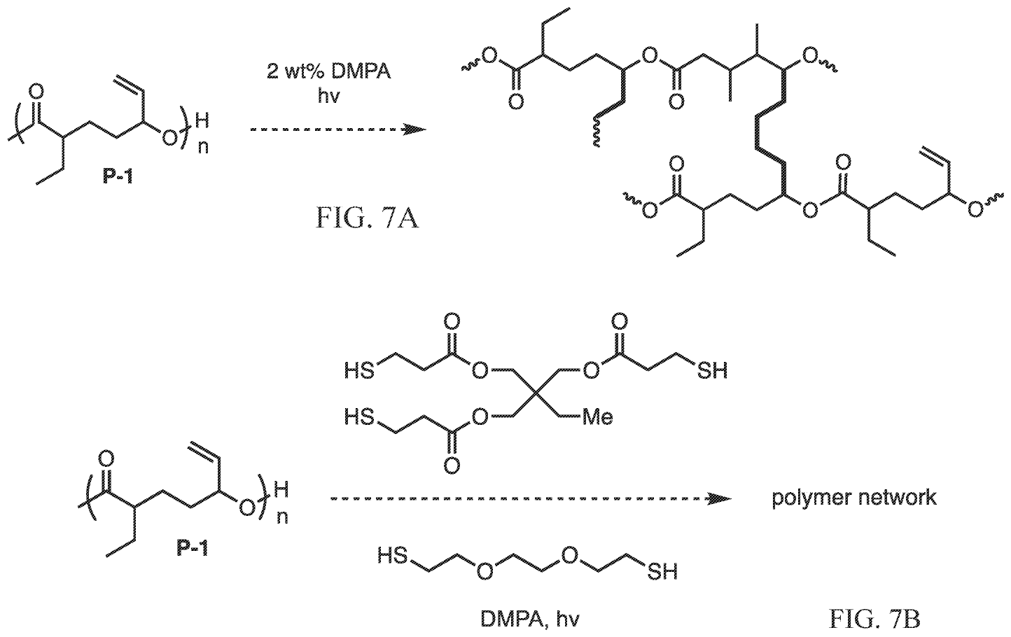

Crosslinking is another modification that utilizes the pendent alkene. The combination of DMPA and UV light can be used to initiate crosslinking of polymer P-1 through its olefins directly, as depicted in FIG. 7A. Additionally, thiol-ene click chemistry can be used to form networks using multi-mercapto coupling agents tri(ethylene glycol)dithiol and trimethylolpropane tris(3-mercaptopropionate) (TMPT), as depicted in FIG. 7B. Increasing the amount of TMPT used as well as complexing the network with metals ions increased the T_{g} values.

### Example 6: Catalyst Screening for EtVP Polymerization

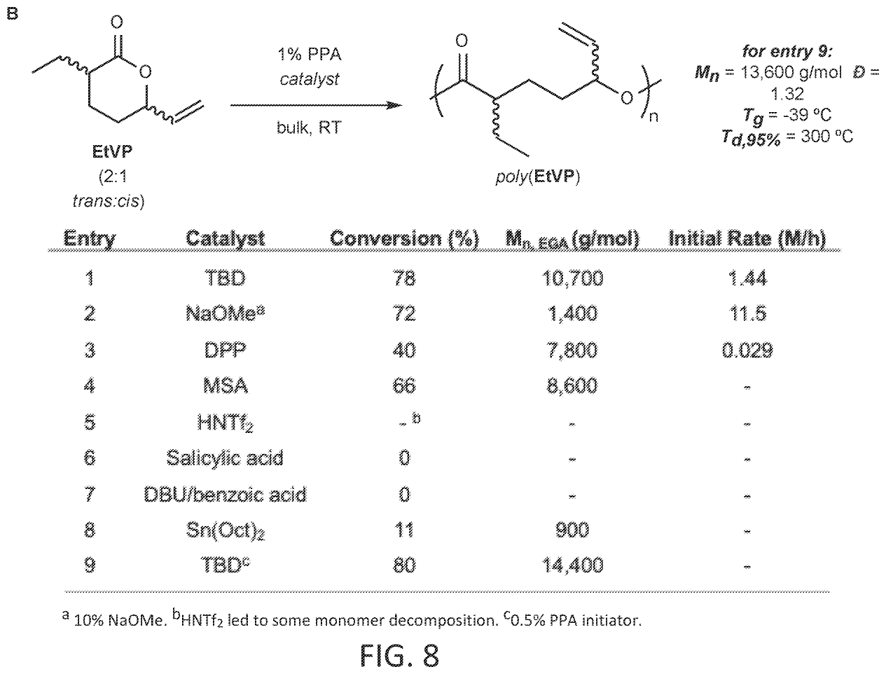

In an N₂ glovebox, 3-phenyl-1-propanol (2.7 mg, 0.02 mmol, 0.01 eq), EtVP (300 mg, 1.95 mmol, 1 eq.), and the desired catalyst (0.1 mmol, 0.05 eq) were added to a 1-dram vial equipped with a stir bar. The polymerization was allowed to stir at room temperature in the glovebox for 3 days. At this point ¹H NMR spectra were taken in CDCl₃; conversion was determined by comparing the monomer peak at 5.86 ppm to the polymer peak at 5.72 ppm, and end-group analysis was carried out using the peak at 5.72 ppm and the initiator peak at 2.68 ppm. See FIG. 8.

Based on these results, 1,5,7-triazabicyclo[4.4.0]dec-5-ene (TBD) was determined to be a good ring-opening polymerization (ROP) catalyst for EtVP. High molar mass poly(EtVP) (13.6 kg/mol, D=1.36) can be synthesized with 0.5% PPA initiator at room temperature. The glass transition temperature (T_{g}) of poly(EtVP) is −38.8° C., making this potentially suitable as a soft block in thermoplastic elastomers. This value is approximately 10° C. higher than comparable monosubstituted 6-lactones (e.g. 6-hexalactone and 6-heptalactone), most likely from impeded chain rotation from the additional substituent.

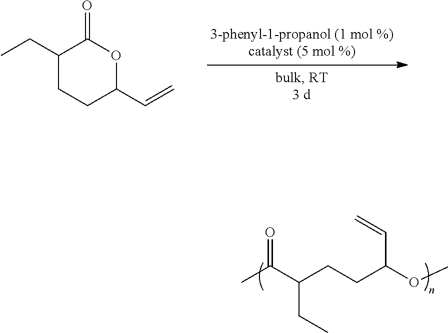

### Example 7: Alternative Procedure for EtVP Polymerization

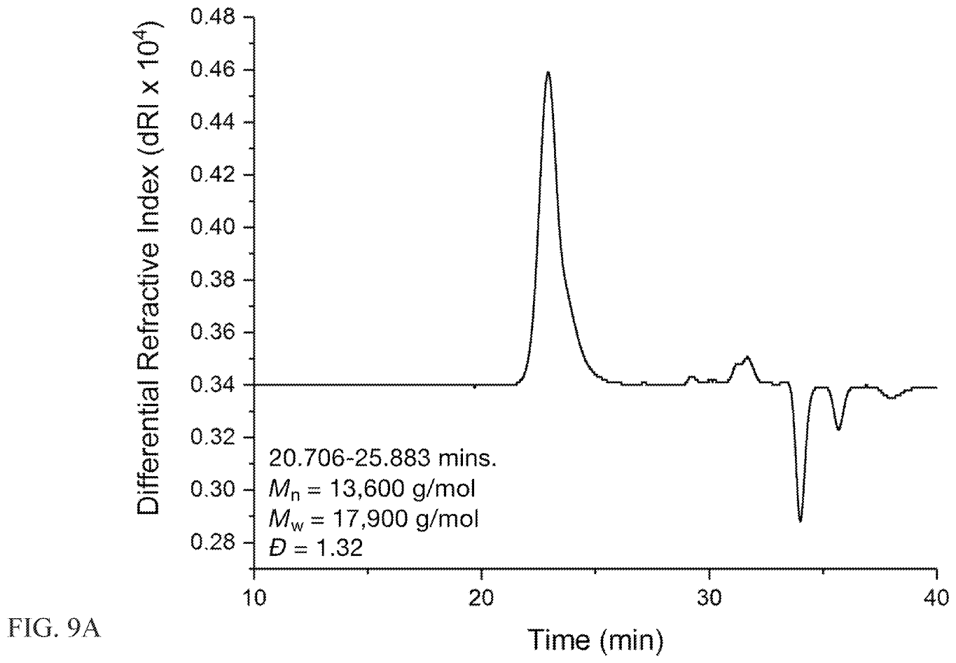

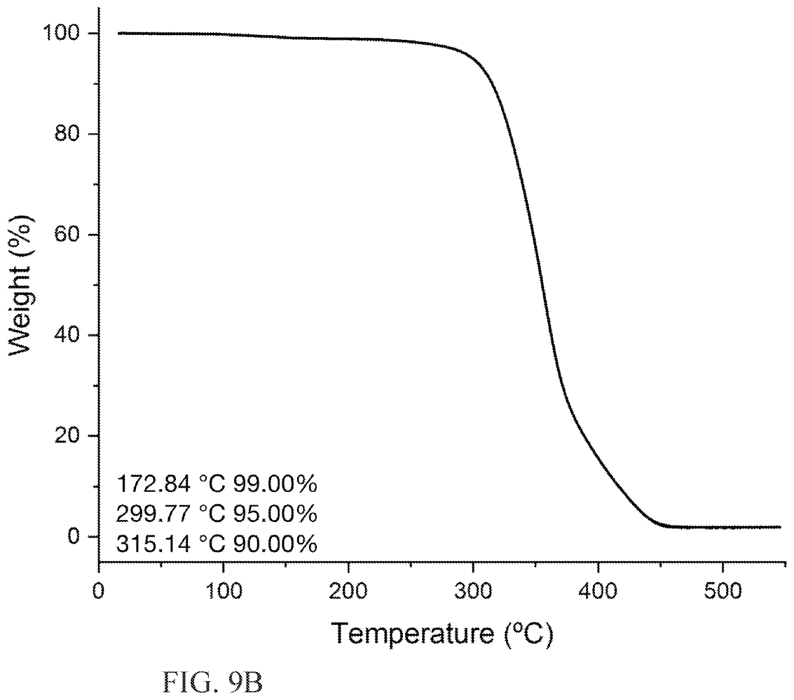

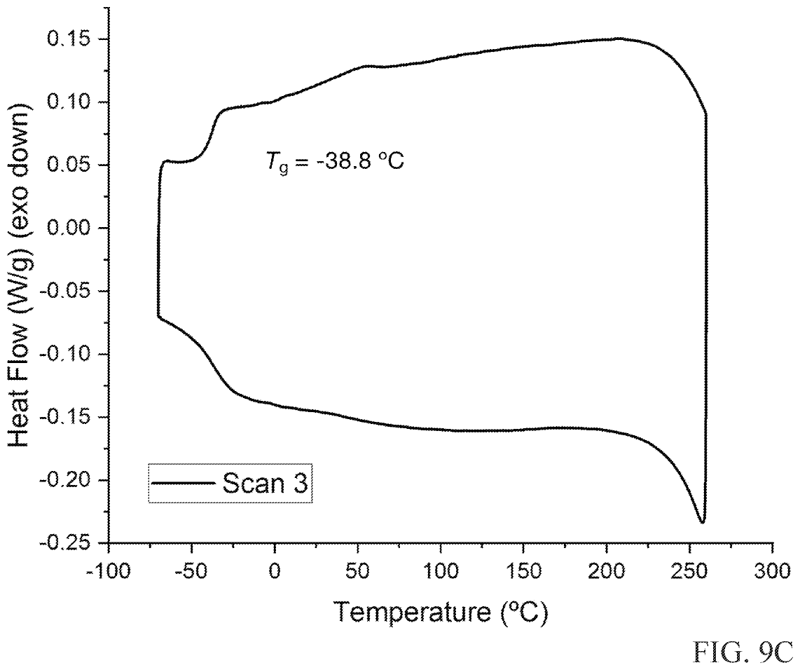

In an N₂ glovebox, 1,5,7-triazabicyclo[4.4.0]dec-5-ene (0.05 eq.) was added to a 20-mL scintillation vial equipped with a stir bar, followed by addition of EtVP (typically 1, 2 or 5 g, 1 eq.) and 3-phenyl-1-propanol (0.005 eq.). The polymerization was allowed to stir at room temperature in the glovebox for 2 or 3 days. At this point a ¹H NMR was taken to confirm approximately 80% conversion had occurred, then the vial was removed from the glovebox and the catalyst was quenched with excess benzoic acid. Purification Method A: After dissolving in minimal CHCl₃ the mixture was slowly pipetted into vigorously stirring hexanes at −78° C. The hexanes was decanted off, then the polymer was dissolved in CHCl₃ and washed 1 time with water and 2 times with brine. The organic phase was then concentrated under reduced pressure at 80° C. for at least 6 hours. Purification Method B: After dissolving in MeCN, the polymerization mixture was vacuum filtered through a silica plug, concentrated under reduced pressure, then vacuum distilled to remove residual monomer. The reaction led to 78% conversion and an Mₙ of 10,700 g/mol (measured by end-group analysis), and kinetic analysis revealed that the initial rate of polymerization was significantly faster (k_{obs}=1.4 M/h) than the corresponding DPP-catalyzed polymerization. ¹H NMR (400 MHz, CDCl₃) δ 5.77-5.67 (m, 1H), 5.26-5.14 (m, 1H), 2.69 (t, 2H, initiator), 2.27 (broad m, 1H), 0.89 (t, 3H); ¹³C NMR (101 MHz, CDCl₃) δ 175.12, 136.42, 117.19, 74.53, 74.04, 47.09, 32.05, 27.58, 25.50, 11.84. See FIG. 9A-9C.

### Example 8: General Procedure for DEP Polymerization

Success with semi-hydrogenated EtVP led to the evaluation of DEP as a candidate for ROP. Conditions were slightly modified from the EtVP ROP conditions (1% benzyl alcohol/5% TBD/−15° C.) led to only 18% conversion of DEP after two days. However, further exploration and optimization of catalysts and conditions led to the discovery that a NaOMe/1-cyclohexyl-3-phenylurea catalyst system was effective for DEP ROP, leading to 70% conversion after one day at room temperature (Mₙ=9.7 kg/mol, D=1.27).

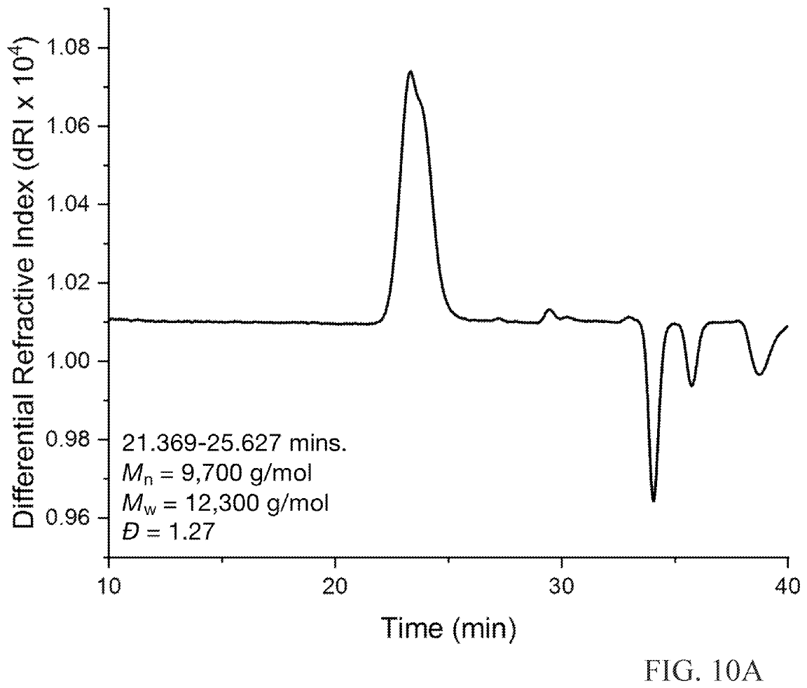

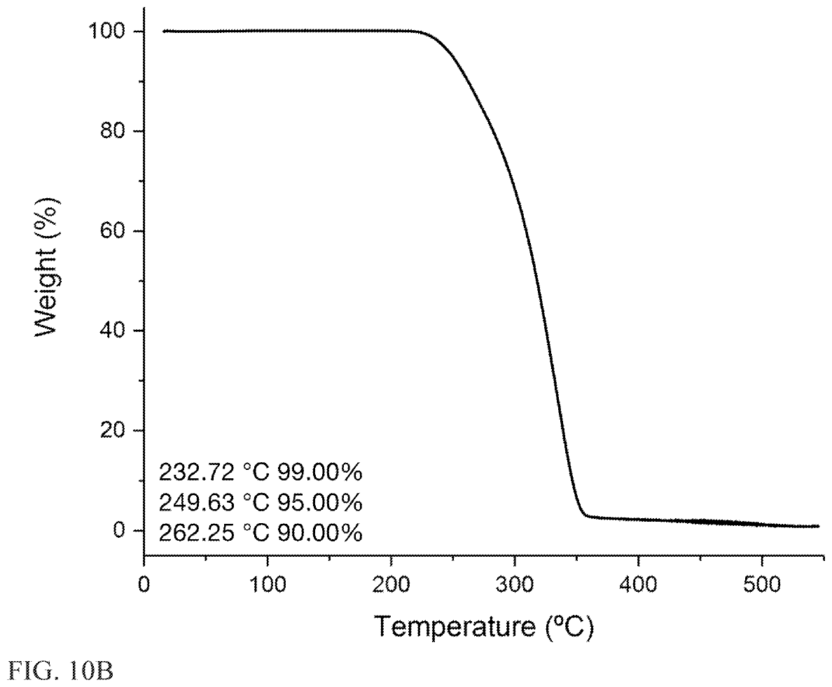

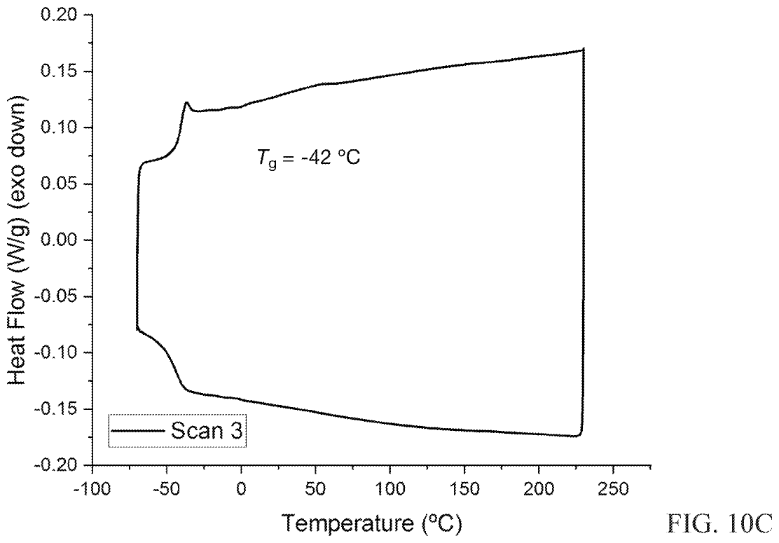

NaOMe (6 mg, 0.11 mmol, 0.03 eq.), 1-cyclohexyl-3-phenylurea (7.2 mg, 0.03 mmol, 0.01 eq.), and DEP (507.2 mg, 3.25 mmol, 1 eq.) were added to a 1-dram scintillation vial equipped with a stir bar in an N₂ glovebox. The reaction mixture was stirred in the glovebox at room temperature for approximately one day, at which point it was removed from the box and quenched with excess benzoic acid. After dissolving in minimal CHCl₃ the mixture was precipitated twice in methanol at −46° C. The methanol was decanted off, then poly(DEP) was dried under vacuum at 80° C. for 16 hours to obtain 94.1 mg of poly(DEP) (18.6% yield). ¹H NMR (400 MHz, CDCl₃) δ 4.85-4.75 (m, 1H), 3.66 (s, 3H), 2.23 (broad s, 1H), 1.68-1.37 (m, 8H), 0.87 (overlapping t, 6H); ¹³C NMR (101 MHz, CDCl₃) δ 175.68, 75.17, 74.69, 47.82, 47.47, 47.38, 47.06, 31.64, 31.49, 31.20, 27.93, 27.52, 26.98, 25.67, 25.63, 25.56, 11.84, 9.65, 9.53. See FIG. 10A-10C.

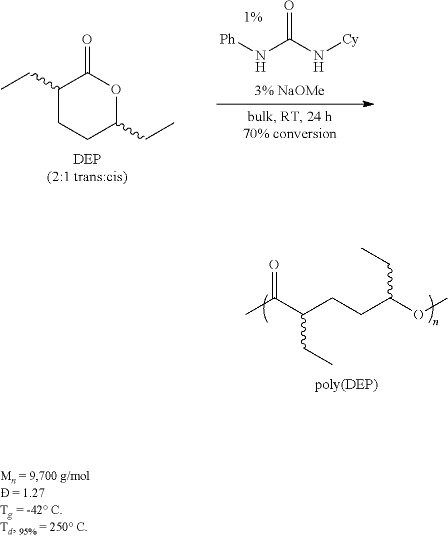

### Example 9: Determination of EtVP Polymerization Thermodynamics and Kinetics

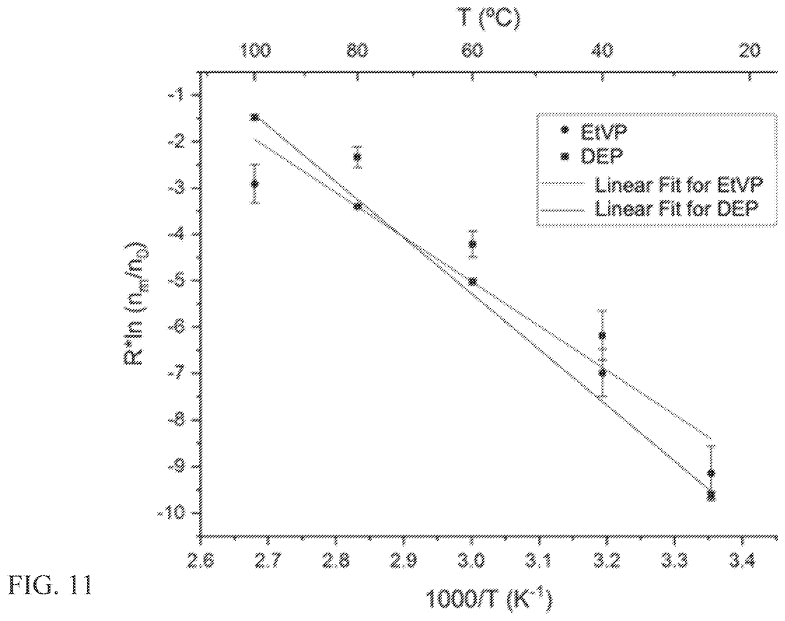

Thermodynamics: Triazabicyclodecene (TBD) (11.3 mg, 0.08 mmol, 0.05 eq.), EtVP (250 mg, 1.6 mmol, 1 eq.), and 3-phenyl-1-propanol (12 mg, 0.05 mmol, 0.05 eq.) were added to a 1-dram scintillation vial equipped with a stir bar in an N₂ glovebox. Following removal from the glovebox, the vials were placed in oil baths at varying temperatures (25-100° C.), with duplicate trials being run for each temperature. Over a period of days, the polymerizations were monitored by ¹H NMR, and reactions were determined to be in equilibrium when conversion was found to be constant between timepoints (within 3%). Conversion was determined by comparing the monomer peak at 5.86 ppm to the polymer peak at 5.72 ppm. The associated thermodynamic parameters were determined using the method outlined by Olsen, Odelius, and Albertsson, Biomacromolecules 2016, 17, 699-709. The thermodynamic parameters of the polymerization of DEP were found in an analogous manner (on a 200 mg DEP scale) using 10% NaOMe and 5% 1-cyclohexyl-3-phenylurea. See FIG. 11.

van't Hoff analysis of TBD-catalyzed polymerization of EtVP revealed thermodynamic parameters of ΔHₚ=−2.26±0.23 kcal/mol and ΔSₚ=−5.48±0.70 cal/mol·K, resulting in a ceiling temperature (T_{c}) of 138° C. (Table 1). Similarly, van′t Hoff analysis of 1-cyclohexyl-3-phenylurea-catalyzed polymerization of DEP revealed ΔHₚ=−2.82±0.23 kcal/mol and ΔSₚ=−7.34±0.68 cal/mol-K, resulting in a ceiling temperature (T_{c}) of 110° C. These low T_{c} values open the possibility of facile chemical recycling (vide infra). Comparison of these values to unsubstituted poly(δ-valerolactone) (ΔHₚ=−2.92 kcal/mol, ΔSₚ=−2.27 cal/mol·K) and poly(δ-hexalactone) (ΔHₚ=−3.3 kcal/mol, ΔSₚ=−5.5 cal/mol·K). The combined polymerization results of EtVP and DEP demonstrate that ROP of disubstituted valerolactones is thermodynamically feasible, although choice of the appropriate catalyst to engender kinetically-accessible polymerizations remains unpredictable.

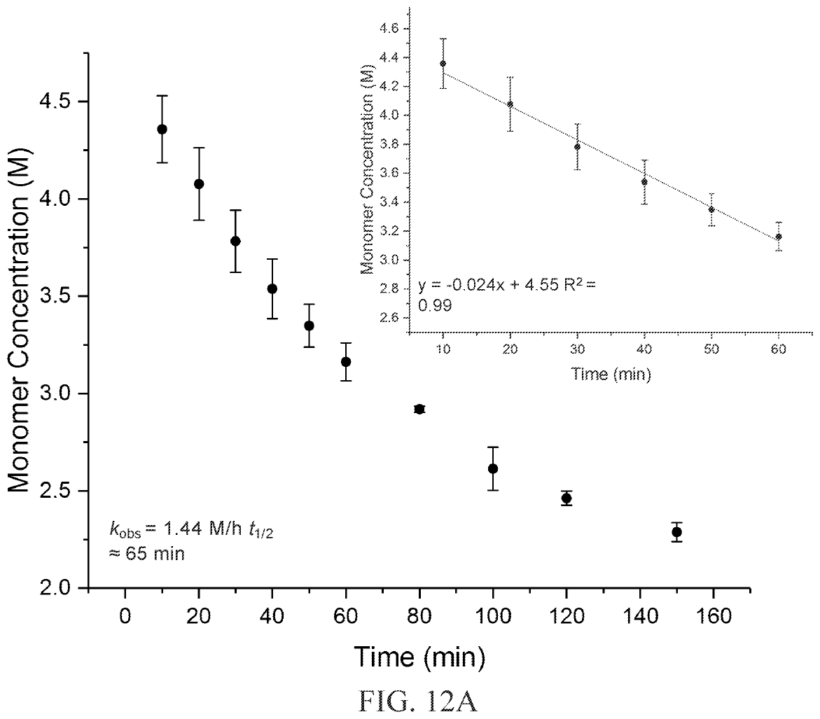

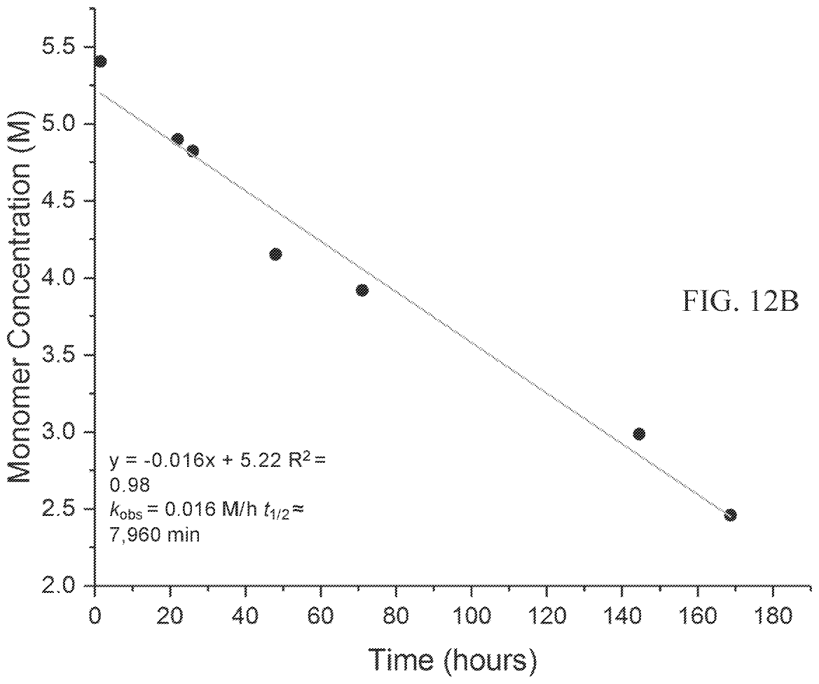

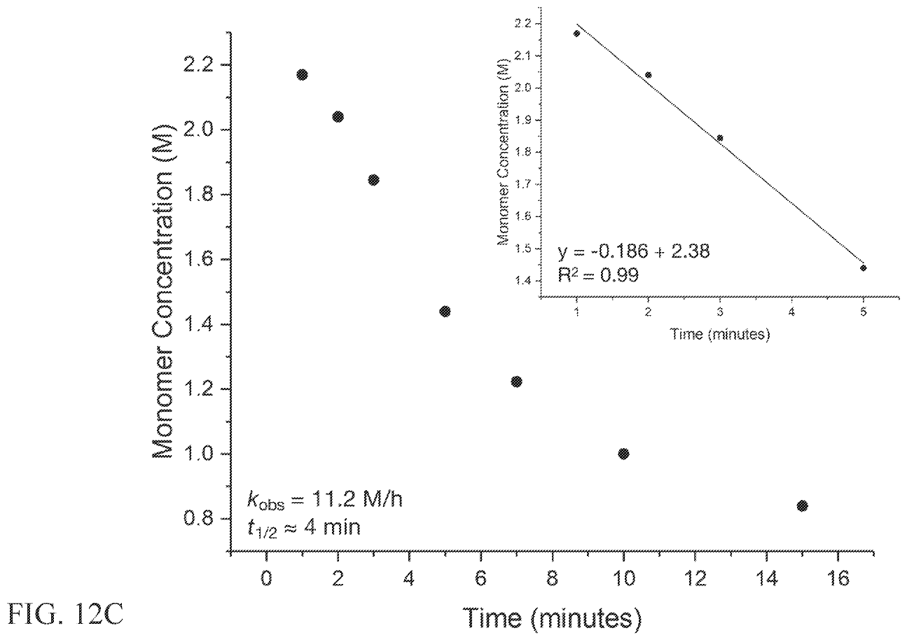

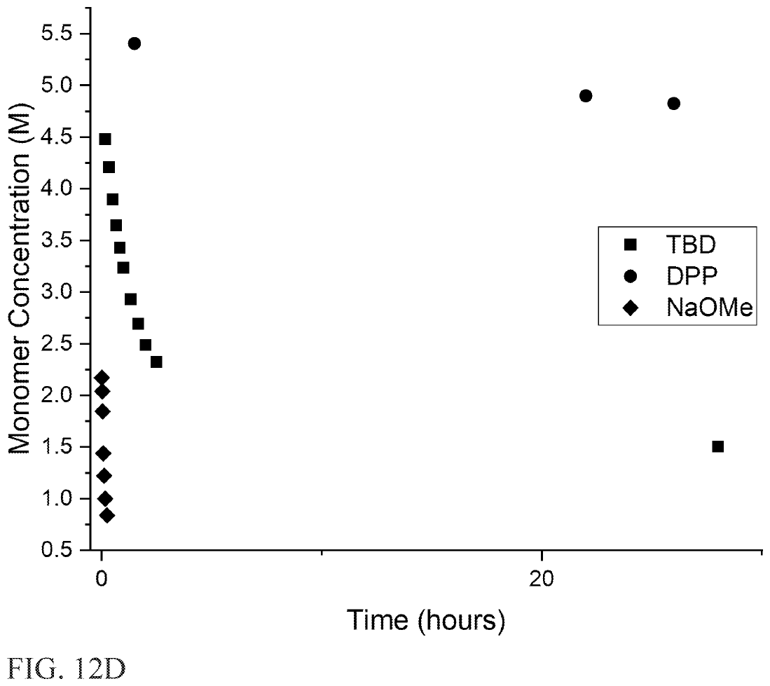

Kinetics: TBD (33.3 mg, 0.24 mmol, 0.05 eq.), EtVP (733 mg, 4.8 mmol, 1 eq.), and 3-phenyl-1-propanol (6.2 mg, 0.05 mmol, 0.01 eq) were added to a 20 mL scintillation vial equipped with a stir bar in an N₂ glovebox. Aliquots were removed and quenched with benzoic acid in CDCl₃ at 10-minute intervals up to one hour, then were taken at 80, 100, 120, and 150 minutes. Monomer concentration was plotted vs. time, and the slope of the linear regime was found to be the k_{obs}. The polymerizations with DPP and NaOMe were run at 5% and 10%, respectively. See FIG. 12A-12D.

### Example 10: TBD-Catalyzed EtVP Epimerization

Diastereomerically-enriched EtVP at a 5.5:1 ratio of trans:cis was attained through flash column chromatography via autocolumn. Distilled EtVP (500 mg, 3.2 mmol) was loaded on the column with a 25 g solid load cartridge and run through two stacked 40 g RediSep® normal-phase silica flash columns. A 95:5 mixture of hexanes to isopropyl alcohol eluent proved to give the broadest EtVP peak after approximately 5 column volumes at 40 mL/min. EtVP was most clearly visible at a 200 nm wavelength. Following the column, the latter half of the EtVP peak (from test tube 15) was separated from the first half and isolated as a 5.5:1 ratio of the diastereomers.

TBD (8.5 mg, 0.06 mmol, 0.05 eq.), 5.5:1 diastereomerically-enriched EtVP (196 mg, 1.3 mmol, 1 eq.), and 3-phenyl-1-propanol (1.5 mg, 0.01 mmol, 0.01 eq) were added to a 1-dram vial equipped with a stir bar in an N₂ glovebox. Aliquots were removed and NMR spectra were taken at 20, 40, 80, 120, 240, and 1440 minutes. Within 20 minutes the monomer epimerized back to the thermodynamic 2:1 trans:cis ratio.

### Example 11: General Procedure for Chain Extension of Poly(EtVP) with L-Lactide

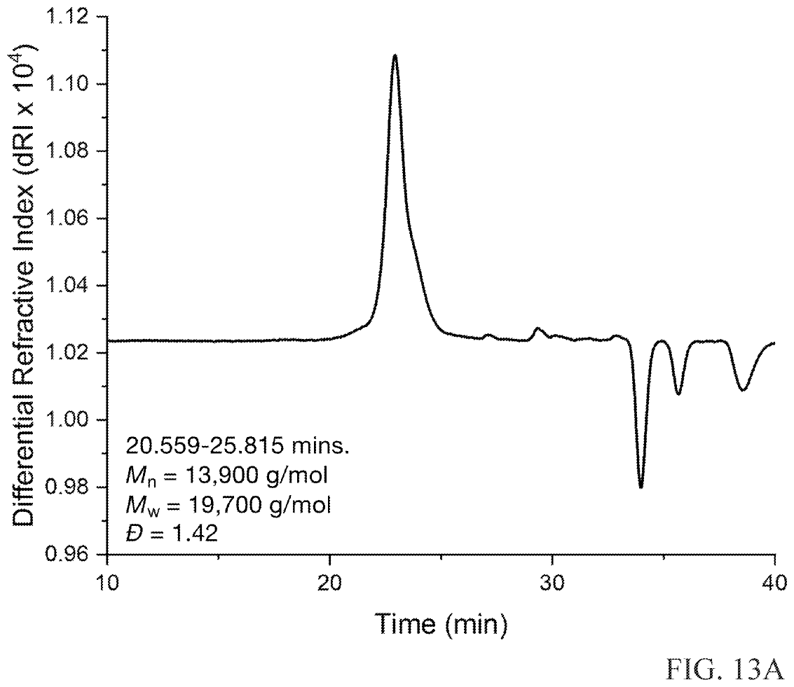

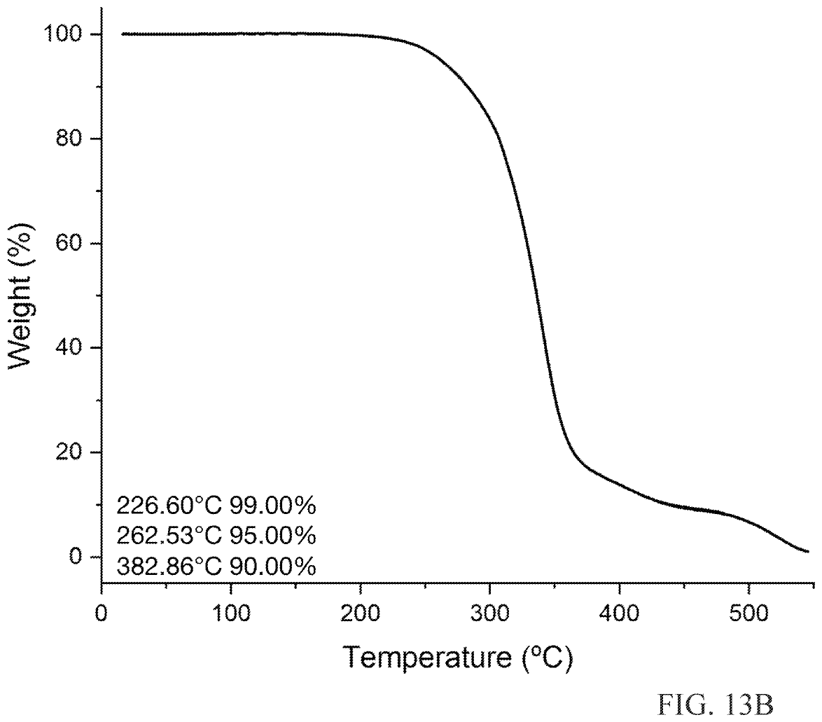

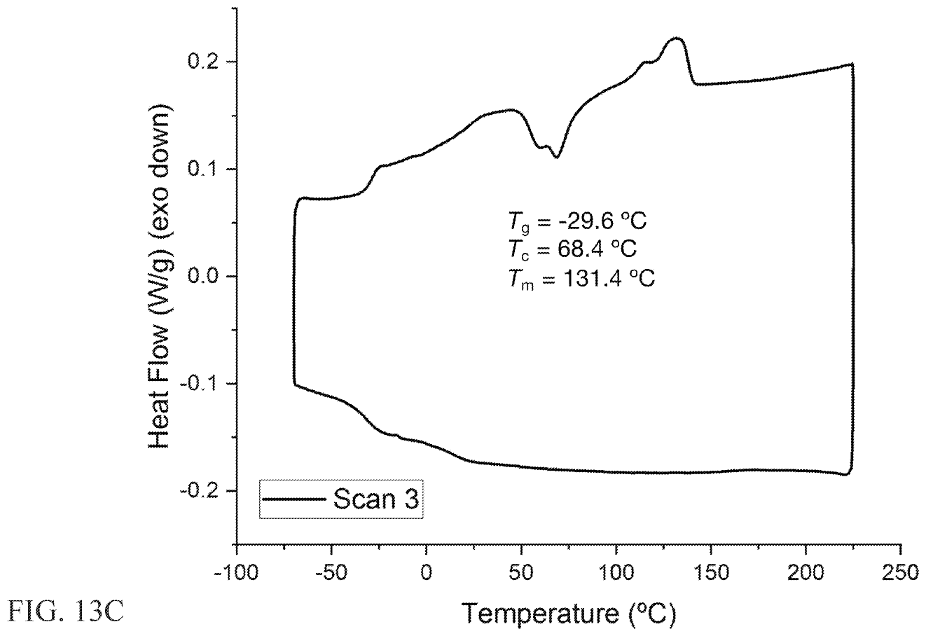

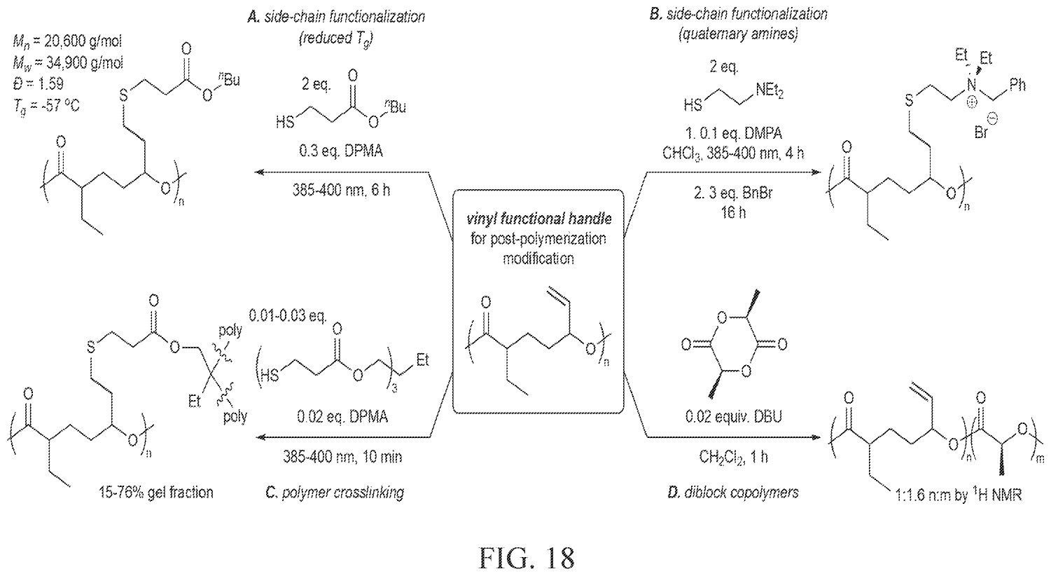

Isolated poly(EtVP) (210.9 mg, 9 kg/mol) was brought into an N₂-filled glovebox in a 20 mL scintillation vial. L-lactide (198.4 mg, 1.4 mmol, 1 eq.), anhydrous DCM (0.9 mL), and 1,8-Diazabicyclo[5.4.0]undec-7-ene (DBU) (3.8 mg, 0.02 mmol, 0.02 eq. to lactide) were then added, and the polymerization was allowed to stir for 1 hour at room temperature. The catalyst was then quenched with excess benzoic acid. The polymerization solution was slowly pipetted into vigorously stirring MeOH at −46° C. and was vacuum filtered. The precipitate was washed off the frit with DCM then concentrated under reduced pressure at 80° C. overnight to give 116.6 mg polymer (28.5% yield). ¹H NMR (400 MHz, CDCl₃) δ 5.76-5.68 (m, 1H), 5.25-5.13 (overlapping d and q, 4H), 2.25 (broad m, 1H), 1.66-1.48 (m, 9H), 0.87 (t, 3H); ¹³C NMR (101 MHz, CDCl₃) δ 174.88, 169.66, 136.36, 117.28, 74.47, 73.99, 69.08, 47.36, 47.04, 32.15, 31.98, 27.52, 27.21, 25.57, 25.44, 16.71, 11.77. See FIG. 13A-13C and FIG. 18.

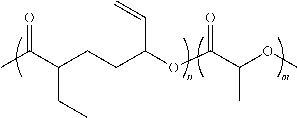

### Example 12: Thiol-Ene Functionalization of Poly(EtVP) with 2-Diethylaminoethanethiol

Poly(EtVP) (228.1 mg, 9 kg/mol, 1 eq.) and 2,2-dimethoxy-2-phenylacetophenone (37.5 mg, 0.15 mmol, 0.1 eq.) were transferred to a 2-dram vial with CHCl₃, and the CHCl₃ was then evaporated. 2-diethylaminoethanethiol-HCl (472.5 mg, 3.3 mmol, 2.3 eq.) and a stir bar were then added to the vial. Stirring and exposure to 9 W, 385-400 nm UV light was then commenced for 3 hours and 20 minutes. The reaction mixture was dissolved in CHCl₃ and washed with saturated NaHCO₃ 2 times. The organic layer was concentrated, dissolved in minimal CHCl₃, then precipitated in 1:1 H₂O:MeOH 3 times. The precipitate was dried under vacuum at 40° C. overnight to obtain 69.9 mg polymer (16.4% yield). ¹H NMR (400 MHz, CDCl₃) δ 4.96-4.90 (m, 1H), 2.63-2.47 (m, 10H), 2.20 (broad m, 1H), 1.81 (broad m, 2H), 1.65-1.52 (m, 6H), 1.03-0.99 (overlapping t, 6H), 0.88 (t, 3H); ¹³C NMR (101 MHz, CD₃OD) δ 176.60, 79.50, 74.13, 53.94, 47.96, 35.33, 33.03, 29.50, 29.09, 28.86, 26.59, 12.56, 11.93. See FIG. 18.

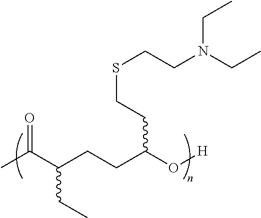

### Example 13: Quaternization of Poly(EtVP-DAT) with Benzyl Bromide

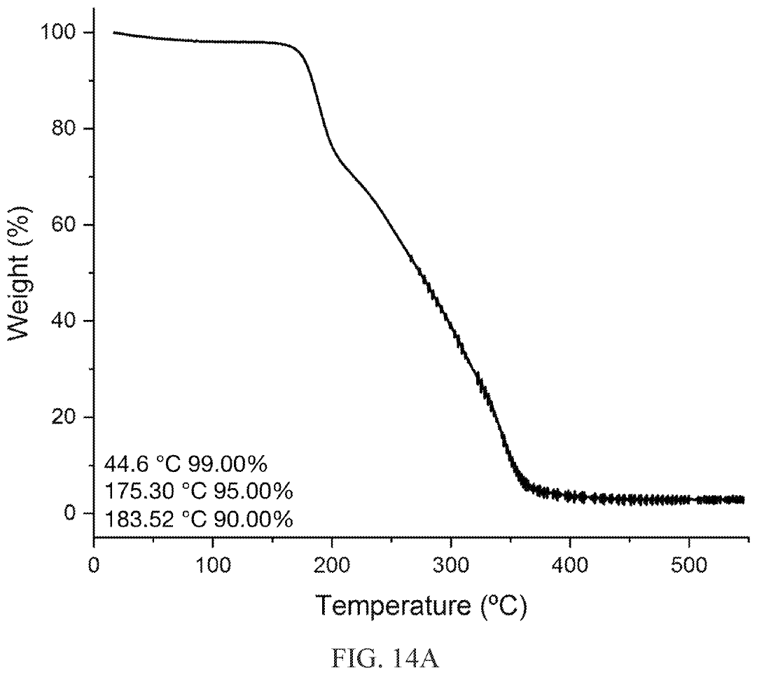

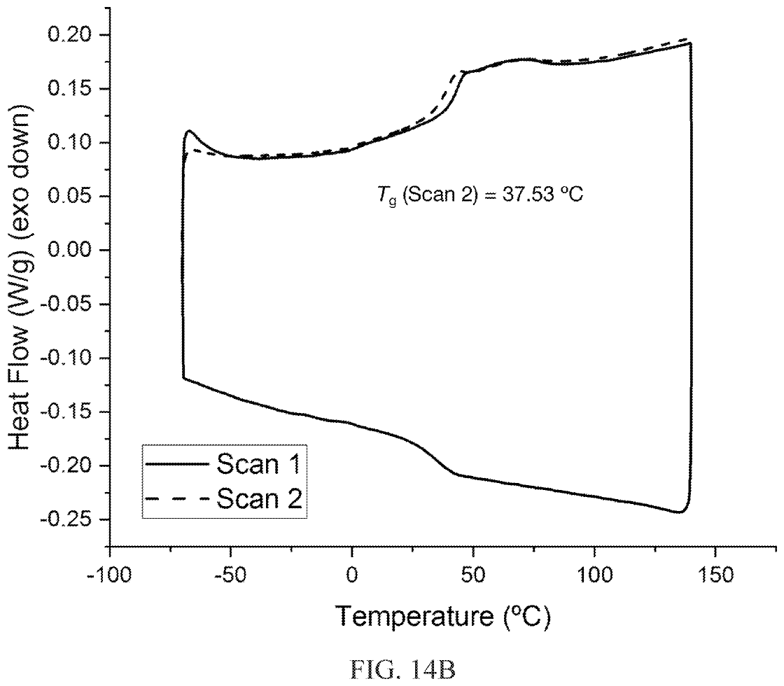

Poly(EtVP-DAT) (279.6 mg, 1 eq.) was added to a 1-dram vial, followed by benzyl bromide (0.44 mL, 3.7 mmol, 3.8 eq.). This was stirred at room temperature for 19 hours, after which the reaction mixture was precipitated 3 times in hexanes. The precipitate was dried under vacuum at 100° C. overnight to obtain 144.8 mg crunchy, solid material (19.7% yield). ¹H NMR (400 MHz, CDCl₃) δ 7.66-7.50 (m, 5H), 5.03 (broad m, 1H), 4.66-4.59 (m, 2H), 3.61-3.37 (m, 6H), 3.11 (broad t, 2H), 2.71 (broad t, 2H), 2.34 (broad m, 1H), 1.93 (broad m, 2H), 1.64-1.45 (m, 12H), 0.93 (broad t, 3H); ¹³C NMR (101 MHz, CD₃OD) δ 177.02, 133.89, 131.89, 130.46, 128.74, 128.53, 79.63, 73.94, 62.51, 58.25, 57.90, 54.59, 35.02, 32.85, 31.42, 29.14, 28.66, 26.70, 26.35, 24.90, 12.42, 9.43, 8.78. See FIGS. 14A and 14B.

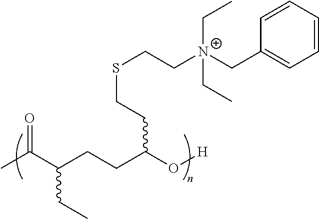

### Example 14: Thiol-Ene Functionalization of Poly(EtVP) with Butyl-3-Mercaptopropionate

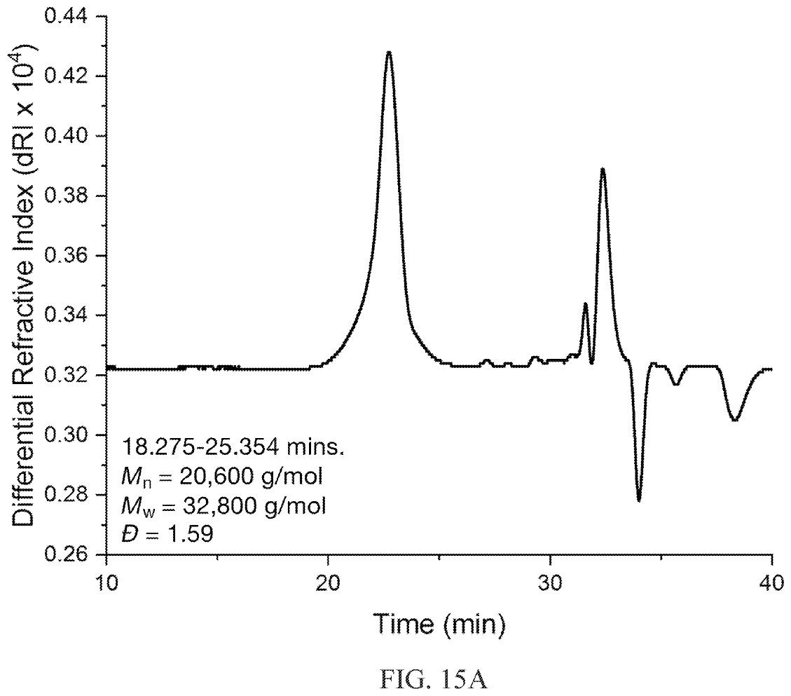

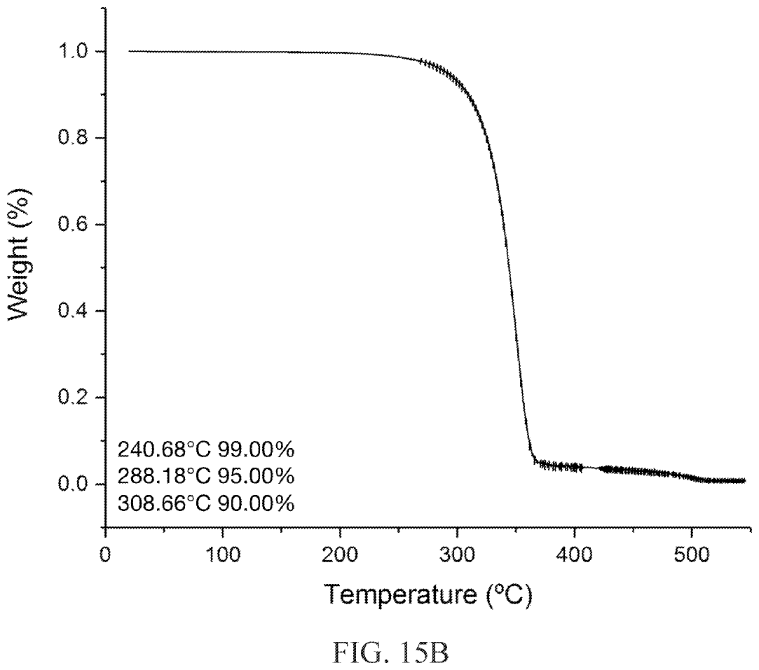

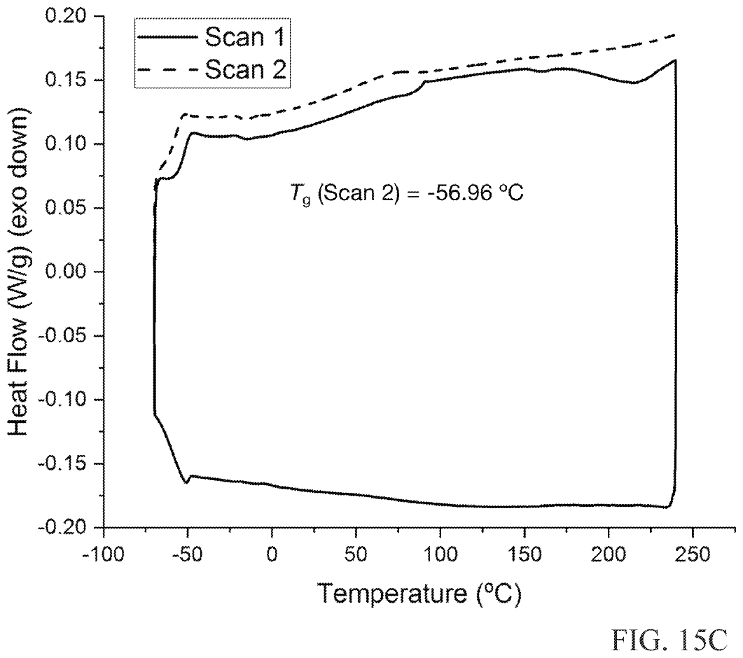

Poly(EtVP) (99 mg, 9.5 kg/mol, 1 eq.) and 2,2-dimethoxy-2-phenylacetophenone (45.2 mg, 0.18 mmol, 0.27 eq.) were transferred to a 2-dram vial with CHCl₃, and the CHCl₃ was then evaporated to prepare a thin film. Butyl-3-mercaptopropionate (0.22 mL, 1.4 mmol, 2.1 eq.) and a stir bar were added to the same vial. Stirring and exposure to 9 W, 385-400 nm UV light was commenced for 5 hours 20 minutes. The reaction mixture was dissolved in minimal CHCl₃ then pipetted into vigorously stirring hexanes at 0° C. After decanting, the polymer was washed 2-3 times with additional hexanes then dried under vacuum at 80° C. for multiple hours to yield 92.6 mg of polymer (45.6% yield). ¹H NMR (400 MHz, CDCl₃) δ 4.96-4.91 (m, 1H), 4.08 (t, 2H), 2.75 (t, 2H), 2.59-2.43 (m, 4H), 2.21 (broad s, 1H), 1.81 (broad s, 2H), 1.64-1.32 (m, 10H), 0.94-0.86 (overlapping t, 6H); ¹³C NMR (101 MHz, CDCl₃) δ 175.46, 172.00, 73.02, 64.69, 47.08, 34.88, 34.18, 32.18, 30.75, 28.07, 27.11, 25.53, 19.25, 13.84, 11.84. See FIG. 15A-15C and FIG. 18.

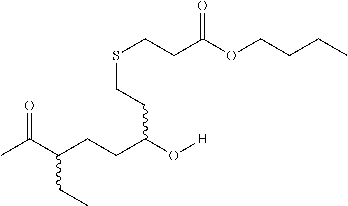 [SMILES: CCCCOC(=O)CCSCCC(O)CCC(CC)C(C)=O]

### Example 15: General Procedure for Poly(EtVP) Crosslinking

Poly(EtVP) (approximately 280 mg, 7.3 kg/mol, 1 eq.), 2,2-dimethoxy-2-phenylacetophenone (5.7 mg, 0.02 mmol, 2 wt %), and trimethylolpropane tris(3-mercaptopropionate) (0.03, 0.02, or 0.01 eq.) were added to a 20 mL scintillation vial, followed by the addition of CHCl₃, which was used to homogenize the mixture, then was evaporated. The mixture was exposed to 9 W, 385-400 nm UV light for 10 minutes. Approximately 50 mg of each network material was set aside for further characterization, and the rest was split into four samples, two for swelling experiments and two for gel fraction experiments. See FIG. 18.

To determine swelling, the material was immersed in 2 mL water for 72 hours, after which it was filtered and weighed. Swelling percentage was determined using the equation below, where Wₛ is the weight of the swollen polymer and W_{d} is the weight of the dry polymer (Dasgupta et. al, Mol. Pharm. 2015, 12, 3479-3489).

$$\% S = \frac{\text{?} - {W}_{d}}{{W}_{d}} \times 100$$

$$\text{?} \text{indicates text missing or illegible when filed}$$

To determine gel fraction, the material was immersed in 2 mL CHCl₃ for 48 hours, after which it was filtered and weighed. Gel fraction was determined using the equation below, where W_{d} is the weight of the initial dry polymer and W_{d2} is the weight after immersion in CHCl₃.

$$\% gel content = \frac{{W}_{d 2}}{{W}_{d 1}} \times 100$$

### Example 16: Hydrolytic Degradation of Poly(EtVP)

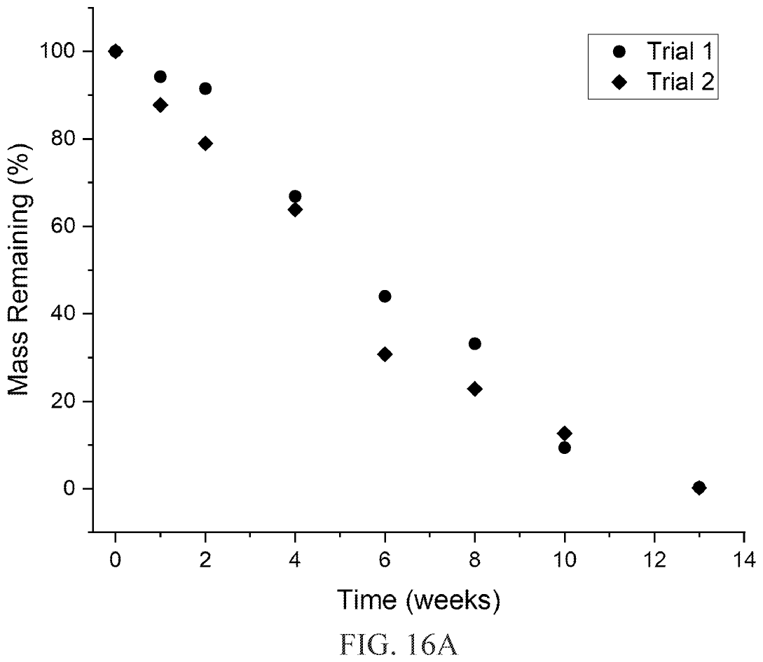

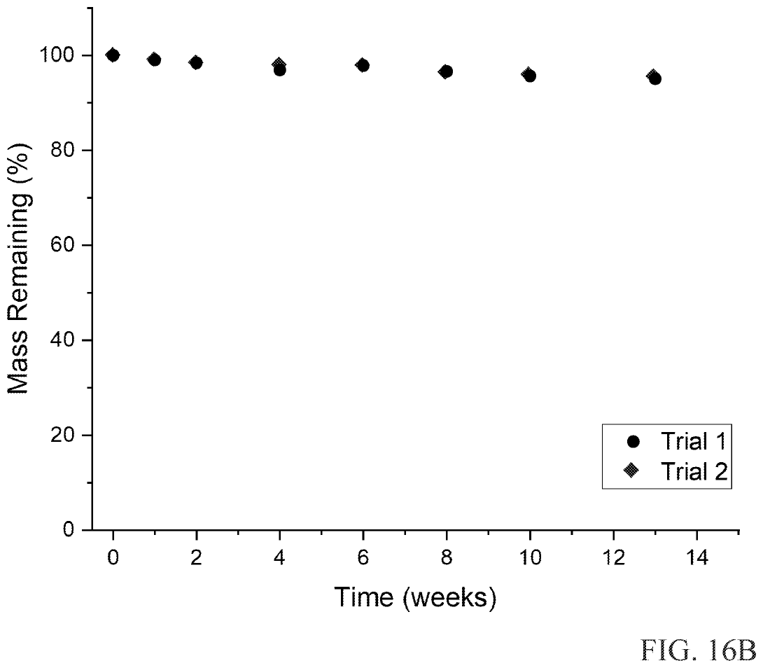

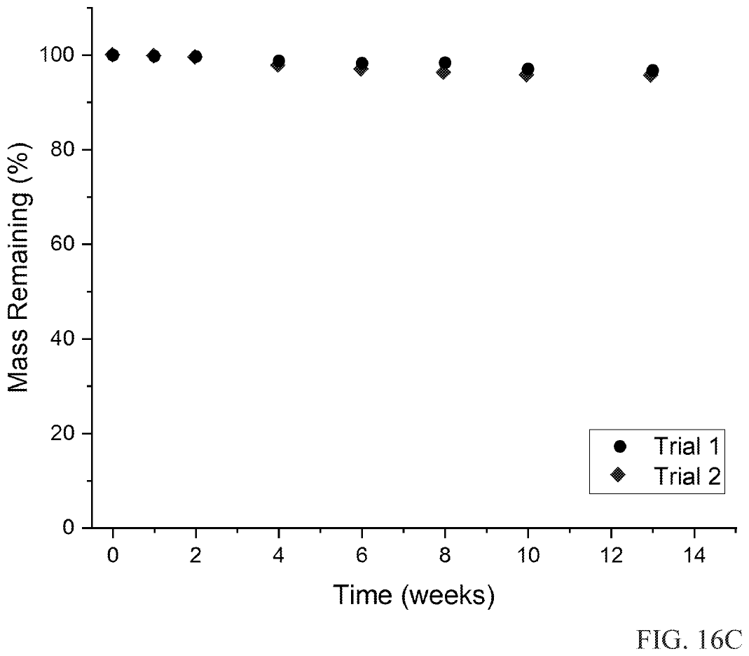

Approximately 100 mg of poly(EtVP) (11 kg/mol) was placed into 6 different 20 mL scintillation vials. Into each vial was added 10 mL of 0.1 M NaOH, 0.1 M HCl, or 0.01 M phosphate-buffered saline (PBS). The vials with NaOH and HCl were stirred at 50° C., and those with PBS were stirred at 37° C. to simulate biological conditions. At each indicated timepoint, the solutions were decanted, and the leftover polymer was washed with water three times then dried under vacuum to a constant weight. See FIG. 16A-16C.

### Example 17: Chemical Recycling of Poly(EtVP) to Monomer

Poly(EtVP) (1.0 g, 9 kg/mol, 1 eq.) and Sn(Oct)₂ (80.5 mg, 0.20 mmol, 0.03 eq.) were added to a 5 mL pear-shaped flask, which was equipped with a simple vacuum distillation apparatus. This was placed under vacuum and heated to 165° C.; after 1 h 40 min, obtained 0.84 g of pure EtVP (84% recovery). The ¹H NMR spectrum of the distillate matched that of EtVP.

### Example 18: Chemical Recycling of Poly(EtVP) with Mixed Polymer Feedstock

Polystyrene, poly(ethylene terephthalate), polypropylene, poly(vinyl chloride), Nylon, high-density polyethylene, polycarbonate, polylactide, polycaprolactone, poly(EtVP) (0.75 g, 9.8 kg/mol), and Sn(Oct)₂ (106 mg, 0.26 mmol, approx. 3 wt %) were added to a 25 mL round-bottom flask equipped with a stir bar (see Table 1). The round-bottom flask was equipped with a short-path vacuum distillation apparatus then heated to 165° C. in an oil bath. 0.24 g of clear distillate was obtained after 6 hours which contained EtVP, lactide, caprolactone, and Sn(Oct)₂.

TABLE 1

| Polymer | Initial Mass (mg) | Weight % |
|---|---|---|
| Poly(EtVP) | 752 | 14 |
| Polystyrene | 511 | 9.5 |
| Poly(ethylene terephthalate) | 515 | 9.6 |
| Poly(vinyl chloride) | 519 | 9.7 |
| High-density | 514 | 9.6 |
| polyethylene | 121 | 2.3 |
| Polypropylene | 517 | 9.6 |
| Nylon | 498 | 9.3 |
| Polycarbonate | 409 | 7.6 |
| Polylactide | 502 | 9.4 |
| Poly(ε-caprolactone) | 510 | 9.5 |

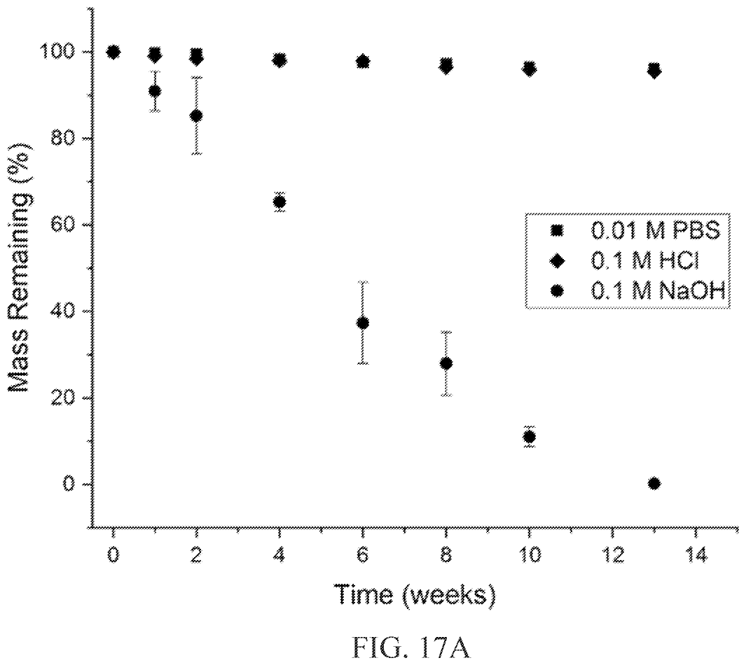

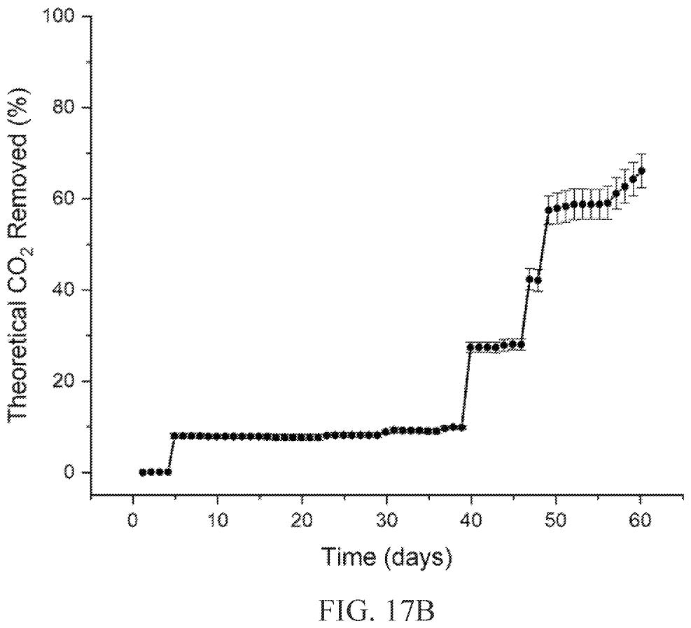

Chemical recyclability is an important feature when considering the overall sustainability of a material. Using a vacuum distillation apparatus, the isolated polymer was exposed to 3% Sn(Oct)₂ at 165° C., from which 84% pure monomer was obtained in less than 2 hours. Chemical recycling of poly(EtVP) from a mixed polymer feedstock was also attempted and, poly(EtVP) was successfully separated from many commodity polymers, but perhaps unsurprisingly co-distilled with lactide and F-caprolactone, which are also susceptible to transesterification. Further, the hydrolytic degradation potential of poly(EtVP) was determined by monitoring mass loss over time in basic (0.1 M NaOH) and acidic (0.1 M HCl) solutions at 50° C. and in 0.01 M phosphate-buffered saline solution (PBS) at 37° C. (FIG. 17A). The polymer almost fully degraded in the basic solution over a period of 13 weeks, compared to only a loss of about 4% in both the HCl and PBS solutions in the same amount of time. Finally, biodegradation studies of this polymer were conducted in an aerobic aqueous environment following OECD-301BB (ready biodegradability) protocol (OECD (1992), Test No. 301: Ready Biodegradability, OECD Guidelines for the Testing of Chemicals, Section 3, OECD Publishing, Paris). These studies showed that the requirements for inherent biodegradability were met, with poly(EtVP) reaching 67.4% of the theoretical CO₂ removed within a 60-day period (FIG. 17B). These studies demonstrated that a CO₂-derived polyester such as poly(EtVP) has the potential for sustainable closed-loop recycling, while also being degradable in the environment in instances where recycling is not possible.

Although this disclosure contains many specific embodiment details, these should not be construed as limitations on the scope of the subject matter or on the scope of what may be claimed, but rather as descriptions of features that may be specific to particular embodiments. Certain features that are described in this disclosure in the context of separate embodiments can also be implemented, in combination, in a single embodiment. Conversely, various features that are described in the context of a single embodiment can also be implemented in multiple embodiments, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

What is claimed is:

1. A method of synthesizing a polyester, the method comprising:
hydrogenating a lactone formed by telomerization of a first 1,3-diene, a second 1,3-diene, and carbon dioxide to yield a hydrogenated lactone; and
polymerizing the hydrogenated lactone in the presence of a catalyst in a ring-opening process to yield a polyester.

2. The method of claim 1, wherein the first 1,3-diene and the second 1,3-diene are the same.

3. The method of claim 1, wherein the first 1,3-diene and the second 1,3-diene are independently butadiene, isoprene, or piperylene.

4. The method of claim 1, wherein the hydrogenated lactone is partially hydrogenated.

5. The method of claim 4, wherein the polyester comprises a pendent olefin.

6. The method of claim 5, further comprising crosslinking the polyester through the pendent olefin to yield a modified polyester.

7. The method of claim 6, wherein crosslinking the polymer through the pendent olefin comprises reacting the pendent olefin with a multi-mercapto coupling agent.

8. The method of claim 5, further comprising modifying the pendent olefin with a thiol-ene click reaction.

9. The method of claim 8, wherein modifying the pendent olefin with the thiol-ene click reaction comprises functionalizing the polyester with a carboxylic acid.

10. The method of claim 8, wherein modifying the pendent olefin with the thiol-ene click reaction comprises functionalizing the polyester with a tertiary amine.

11. The method of claim 10, further comprising quaternizing the amine to yield a modified polyester with antibacterial properties.

12. The method of claim 1, wherein the catalyst is an acid catalyst or an organocatalyst.

13. The method of claim 1, wherein the catalyst comprises one or more of diphenyl phosphate, sodium methoxide, triazabicyclodecene, bistriflimide, methane-sulfonic acid, and diaza-bicycloundecene.

14. The method of claim 1, wherein the polymerizing occurs at a temperature between about 0° C. and about 166° C.

15. The method of claim 1, further comprising synthesizing the lactone.

16. The method of claim 15, wherein synthesizing the lactone comprises capturing carbon dioxide from the atmosphere.

17. The polyester of claim 1.

18. The polyester of claim 17, wherein the polyester is represented by one of the following structures: 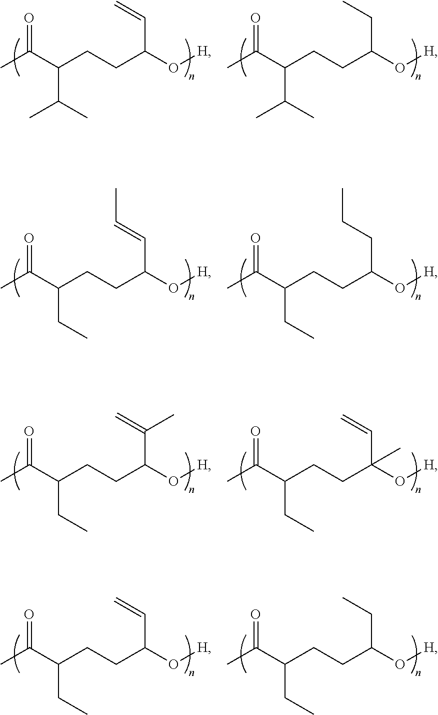
wherein n is an integer.

19. The polyester of claim 17, wherein n is in a range between about 2 and about 200.

20. The modified polyester of claim 6.

21. A method of synthesizing a lactone, the method comprising:
telomerizing piperylene with carbon dioxide to yield the lactone.

22. The method of claim 21, wherein the lactone is represented by one of the following structures:

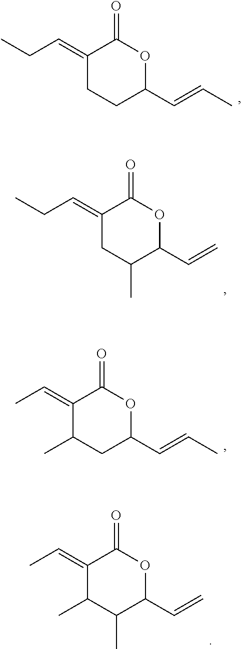

23. The lactone of claim 21.

24. A method of synthesizing a polyester, the method comprising:
hydrogenating a lactone formed by the method of claim 21 to yield a hydrogenated lactone; and
polymerizing the hydrogenated lactone in the presence of a catalyst in a ring-opening process to yield a polyester.

25. The polyester of claim 24.
